(19) United States
(12) Patent Application Publication
(10) Pub. No.: US 2025/0387118 A1
(43) Pub. Date: Dec. 25, 2025

(54) SURGICAL INSTRUMENT

(71) Applicant: LIVSMED INC., Seongnam-si (KR)

(72) Inventors: Woo Jung CHOI, Siheung-si (KR); Han Vit GIL, Suwon-si (KR)

(21) Appl. No.: 19/242,807

(22) Filed: Jun. 18, 2025

(30) Foreign Application Priority Data
Jun. 19, 2024 (KR) 10-2024-0079491

(51) Int. Cl. A61B 17/072; A61B 17/00

(52) U.S. Cl. CPC A61B 17/072; A61B 2017/00398; A61B 2017/07271

(57) ABSTRACT

Provided is a surgical instrument including an end tool configured to perform a rotation in at least one direction, a drive wire having a first wire and a second wire that control the rotation of the end tool, a power generation part having at least one motor configured to generate a power to drive the end tool, and a power transmission part connected to the end tool and configured to transmit the power generated by the power generation part to the end tool, wherein the power transmission part includes a first power transmission unit configured to linearly move the first wire in a longitudinal direction of the first wire by driving of the at least one motor, and a second power transmission unit configured to linearly move the second wire in a longitudinal direction of the second wire by the driving of the at least one motor.

### CROSS-REFERENCE TO RELATED APPLICATION

This application is based on and claims priority under 35 USC § 119 to Korean Patent Application No. 10-2024-0079491, filed on Jun. 19, 2024, in the Korean Intellectual Property Office, the disclosure of which is incorporated by reference herein in its entirety.

### BACKGROUND

### 1. Field

The present disclosure relates to a surgical instrument, and more particularly, to a surgical instrument that is mountable on a robot arm or operable manually for use in laparoscopic surgery or various other surgical procedures.

### 2. Description of the Related Art

Medically, surgery refers to the treatment of diseases by cutting, slitting, or manipulating the skin, mucous membranes, or other tissues using medical devices. In particular, open surgery, which cuts and opens the skin of a surgical site and cures, shapes, or removes an organ therein, may cause bleeding, side effects, patient pain, scars, or the like. Accordingly, recently, surgery performed by inserting only a medical device, for example, laparoscopic surgical instrument, microsurgical microscope, and the like by forming a predetermined hole in the skin or surgery using a robot has been spotlighted as an alternative.

A surgical instrument is a tool equipped with an end tool provided on one end of a shaft that passes through a hole drilled in the skin, and is manipulated by a medical doctor by hand using a predetermined driving part or by a robot arm to perform surgery at the surgical site. The end tool provided on the surgical instrument performs a rotational motion, a gripping motion, a cutting motion, or the like through a predetermined structure.

The background art described above is technical information retained by the present inventors in order to derive the present disclosure or obtained by the present inventors in the process of deriving the present disclosure, and thus is not necessarily known art disclosed to the general public before the filing of the present application.

### SUMMARY

The present disclosure is directed to providing a surgical instrument that is mountable on a robot arm or operable manually for use in laparoscopic surgery or various other surgical procedures, the surgical instrument being capable of manipulating wires by converting a rotational motion of a motor into a linear motion.

The present disclosure is directed to providing a surgical instrument including an end tool configured to be rotatable in at least one direction, a drive wire having a first wire and a second wire that control the rotation of the end tool, a power generation part having at least one motor configured to generate a power to drive the end tool, and a power transmission part connected to the end tool and configured to transmit the power generated by the power generation part to the end tool, wherein the power transmission part includes a first power transmission unit configured to linearly move the first wire in a longitudinal direction of the first wire by driving of the at least one motor, and a second power transmission unit configured to linearly move the second wire in a longitudinal direction of the second wire by the driving of the at least one motor.

In an embodiment of the present disclosure, when the at least one motor is driven, the first wire and the second wire may be linearly moved in opposite directions by the first power transmission unit and the second power transmission unit.

In an embodiment of the present disclosure, the first power transmission unit may include a first lead screw configured to be rotated by the driving of the at least one motor and having a first thread, and a first linear movement guide connected to the first wire, threadedly engaged with the first lead screw, and configured to move linearly in response to the rotation of the first lead screw, and the second power transmission unit may include a second lead screw configured to be rotated by the driving of the at least one motor and having a second thread, and a second linear movement guide connected to the second wire, threadedly engaged with the second lead screw, and configured to move linearly in response to the rotation of the second lead screw.

In an embodiment of the present disclosure, the power generation part may include a main gear coupled to the at least one motor and configured to be rotatable, the first power transmission unit may further include a first sub-gear, to which one end of the first lead screw is connected and which is engaged with the main gear at one side of the main gear, and the second power transmission unit may further include a second sub-gear, to which one end of the second lead screws is connected and which is engaged with the main gear at another side of the main gear.

In an embodiment of the present disclosure, the first thread and the second thread may be formed in opposite directions.

In an embodiment of the present disclosure, the surgical instrument may further include a connection part positioned between the power generation part and the end tool and having a shaft in which the drive wire is accommodated, wherein the connection part may include a first wire coupling member having one end connected to the first wire and configured to move linearly while maintaining contact between the other end and the first linear movement guide, and a second wire coupling member having one end connected to the second wire and configured to move linearly while maintaining contact between the other end and the second linear movement guide.

In an embodiment of the present disclosure, the connection part may further include a direction-changing member positioned between the first wire coupling member and the second wire coupling member and configured to switch movement directions of the first wire and the second wire.

In an embodiment of the present disclosure, the direction-changing member may be provided as an auxiliary pulley, and the first wire and the second wire may extend to the end tool while being wound around the auxiliary pulley.

In an embodiment of the present disclosure, the first wire coupling member may have one end fixed to the first wire and include first gear teeth formed in a longitudinal direction of the first wire coupling member, the second wire coupling member may have one end fixed to the second wire and include second gear teeth formed in a longitudinal direction of the second wire coupling member, and the direction-changing member may be provided as an auxiliary gear engaged with the first gear teeth and the second gear teeth.

In an embodiment of the present disclosure, the connection part may further include a first wire fixing member fixed to the first wire and configured to connect the first wire coupling member to the first wire, and a second wire fixing member fixed to the second wire and configured to connect the second wire coupling member to the second wire.

In an embodiment of the present disclosure, the power generation part may include a motor pack having a motor housing and a plurality of drive motors positioned inside the motor housing, wherein the motor pack may include a pitch drive motor configured to generate a power to enable the end tool to perform a pitch motion, and a yaw drive motor configured to generate a power to enable the end tool to perform a yaw motion.

In an embodiment of the present disclosure, the power generation part may further include a roll rotation unit configured to roll-rotate the motor pack, wherein the roll rotation unit may include a roll drive motor configured to generate a power to roll-rotate the motor pack, a roll drive gear connected to the roll drive motor, and a rotation guide unit connected to the motor housing, engaged with the roll drive gear, and configured to roll-rotate the motor pack by driving of the roll drive motor.

In an embodiment of the present disclosure, the power transmission part may further include a yaw power transmission part configured to control a movement of a yaw drive wire by driving of the yaw drive motor, and a pitch power transmission part configured to control a movement of a pitch drive wire by driving of the pitch drive motor.

In an embodiment of the present disclosure, the end tool may include an operation member moving in a longitudinal direction of the end tool, and the motor pack may further include a firing drive motor configured to generate a power to linearly move the operation member.

In an embodiment of the present disclosure, the power transmission part may further include a firing power transmission part configured to control a movement of a firing drive wire by driving of the firing drive motor.

The present disclosure is also directed to providing a handle of a surgical instrument including a power generation part having at least one motor that generates a power to drive an end tool configured to be rotatable in at least one direction, and a power transmission part configured to linearly move a first wire and a second wire, which control the rotation of the end tool, and transmit the power generated by the power generation part to the end tool, wherein the power transmission part includes a first power transmission unit configured to linearly move the first wire in a longitudinal direction of the first wire by driving of the at least one motor, and a second power transmission unit configured to linearly move the second wire in a longitudinal direction of the second wire by the driving of the at least one motor.

In an embodiment of the present disclosure, when the at least one motor is driven, the first wire and the second wire may be linearly moved in opposite directions by the first power transmission unit and the second power transmission unit.

In an embodiment of the present disclosure, the first power transmission unit may include a first lead screw configured to be rotated by the driving of the at least one motor and having a first thread, and a first linear movement guide connected to the first wire, threadedly engaged with the first lead screw, and configured to move linearly in response to the rotation of the first lead screw, and the second power transmission unit may include a second lead screw configured to be rotated by the driving of the at least one motor and having a second thread, and a second linear movement guide connected to the second wire, threadedly engaged with the second lead screw, and configured to move linearly in response to the rotation of the second lead screw.

The present disclosure is also directed to providing a connection part of a surgical instrument, the connection part including a shaft positioned between an end tool configured to be rotatable in at least one direction and a power generation part configured to provide a power to the end tool, the shaft accommodating a first wire and a second wire that control the rotation of the end tool, a first wire coupling member positioned inside the shaft and having one end connected to the first wire, and a second wire coupling member positioned inside the shaft and having one end connected to the second wire, wherein the first wire coupling member and the second wire coupling member linearly move the first wire and the second wire in opposite directions while being linearly moved by driving of a motor of the power generation part.

In an embodiment of the present disclosure, the connection part may further include a direction-changing member positioned between the first wire coupling member and the second wire coupling member and configured to switch movement directions of the first wire and the second wire.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view illustrating a surgical instrument according to an embodiment of the present disclosure;

FIG. 2 is a side view of the surgical instrument of FIG. 1;

FIG. 3 is a perspective view illustrating an end tool according to an embodiment of the present disclosure;

FIG. 4 is a perspective view illustrating the end tool of FIG. 3 with an end tool hub and a pitch hub removed;

FIG. 5 is a perspective view illustrating a first jaw and a cartridge of the end tool of FIG. 3;

FIG. 6 is an exploded perspective view of the cartridge of FIG. 5;

FIG. 7 is a perspective cross-sectional view for describing an internal structure of the cartridge of FIG. 6;

FIG. 8 is a perspective view illustrating a manipulation part and a power generation part according to an embodiment of the present disclosure;

FIG. 9 is a magnified perspective view illustrating the power generation part of FIG. 8;

FIG. 10 is a cross-sectional view for describing a gear structure of the power generation part of FIG. 8;

FIGS. 11 to 13 are conceptual diagrams illustrating a power transmission structure of the surgical instrument according to an embodiment of the present disclosure and operations of the end tool;

FIGS. 14 and 15 are perspective views illustrating a plurality of power transmission parts provided in the surgical instrument according to an embodiment of the present disclosure;

FIGS. 16 and 17 are perspective views illustrating some components of a yaw power transmission part of FIG. 14;

FIGS. 18 and 19 are perspective views illustrating a plurality of power transmission parts provided in a surgical instrument according to another embodiment of the present disclosure; and

FIG. 20 is a perspective view illustrating some components of a yaw power transmission part of FIG. 18.

### DETAILED DESCRIPTION

Hereinafter, the following embodiments will be described in detail with reference to the accompanying drawings. When describing with reference to the drawings, identical or corresponding components will be assigned the same reference numerals and duplicate descriptions thereof will be omitted.

Since various transformations can be made to these embodiments, specific embodiments will be illustrated in the drawings and described in detail in the detailed description. The effects and features of the present embodiments and the accompanying methods thereof will become apparent from the following description of the contents, taken in conjunction with the accompanying drawings. However, the present embodiments are not limited to the embodiments disclosed below, but may be implemented in various forms.

In describing the present disclosure, a detailed description of known related arts will be omitted when it is determined that the gist of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms The terms are only used to distinguish one component from another.

In the following embodiments, terms such as “include” or “have” means that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another members from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

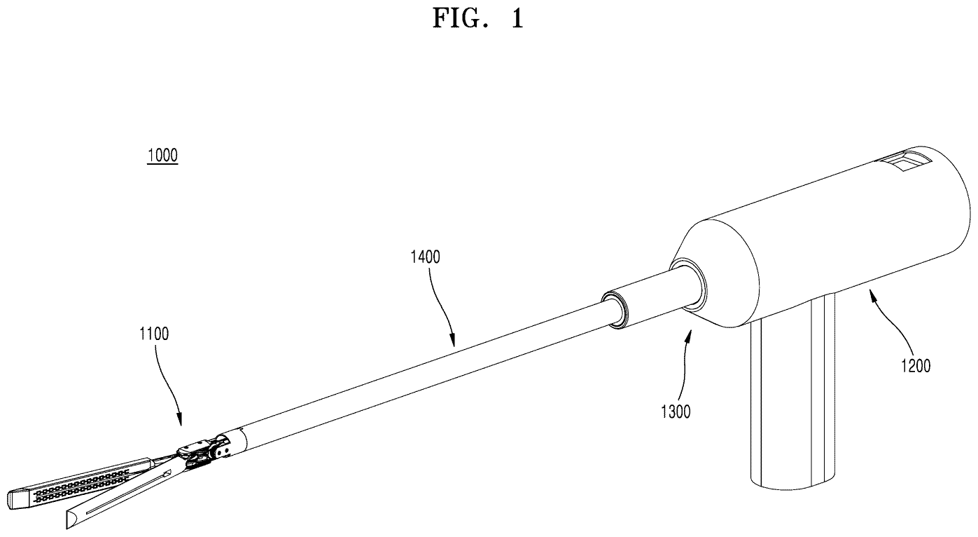

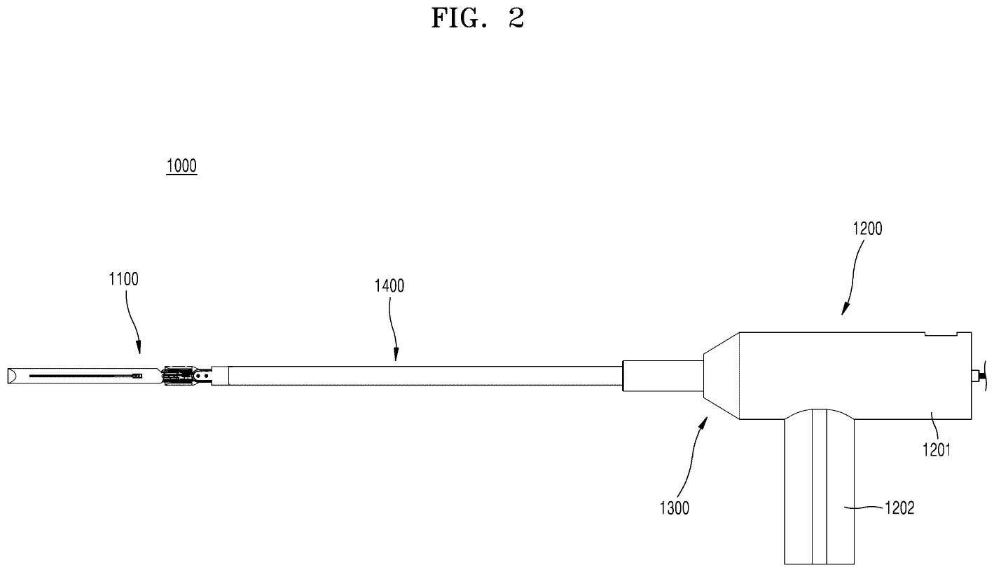

FIG. 1 is a perspective view illustrating a surgical instrument 1000 according to an embodiment of the present disclosure, and FIG. 2 is a side view of the surgical instrument 1000 of FIG. 1.

Referring to FIGS. 1 and 2, the surgical instrument 1000 according to an embodiment of the present disclosure may include an end tool 1100, a manipulation part 1200, a power transmission part 1300, and a connection part 1400.

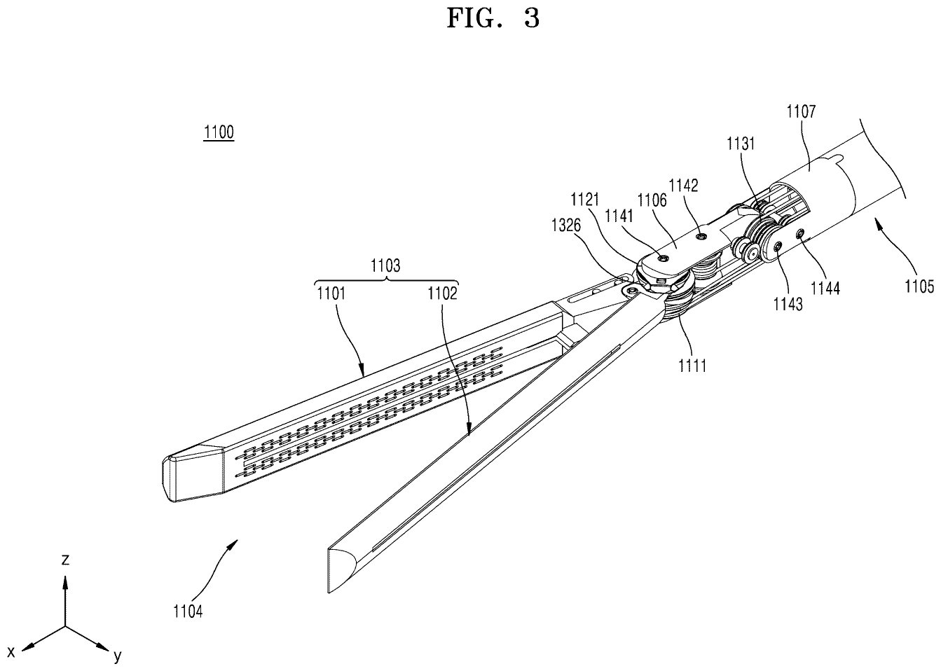

The end tool 1100 is formed on one end portion of the connection part 1400, and performs necessary motions for surgery by being inserted into a surgical site. As an example of the end tool 1100 described above, a pair of jaws 1103 for performing a grip motion may be used as shown in FIG. 3 or the like. The above-described end tool 1100 is connected to the manipulation part 1200 by the power transmission part 1300 and the connection part 1400, which will be described later, and receives a driving force of the manipulation part 1200 through the power transmission part 1300 and/or the connection part 1400 to perform a motion necessary for surgery, such as gripping, cutting, suturing, or the like. However, the concept of the present disclosure is not limited thereto, and various devices for performing surgery may be used as the end tool 1100. For example, in the following description, for convenience of description, the end tool 1100 used as a stapler is described by way of example, but the present disclosure is not limited thereto, and configurations such as a surgical clamp, a surgical grasper, a vessel scaler, and a monopolar electrocautery may also be used as the end tool.

A user may operate the end tool 1100 by manipulating the manipulation part 1200. For example, the manipulation part 1200 is a component for the user to input signals to control motions of the end tool 1100. That is, the manipulation part 1200 may be described as a component that receives signals from the user to control the motions of the end tool 1100. Here, the signals for controlling the motions of the end tool 1100 may correspond to mechanical manipulations such as pressing a button or switch, or rotating or moving a particular member, and may also be electrical signals generated by such mechanical manipulations, but the present disclosure is not limited thereto. The manipulation part 1200 is provided as an interface to be directly controlled by a medical doctor, for example, provided in a gun shape, a tongs shape, a stick shape, a lever shape, or the like, and when the medical doctor controls the manipulation part 1200, the end tool 1100, which is connected to the corresponding interface and inserted into the body of a surgical patient, performs a certain motion, thereby performing surgery. Here, the manipulation part 1200 is illustrated in FIG. 1 as being formed in a gun shape, but the concept of the present disclosure is not limited thereto, and various types of manipulation parts that can be connected to the end tool 1100 and manipulate the end tool 1100 may be possible.

As an example, the manipulation part 1200 may be a separate component or module separated from a drive module of the surgical instrument 1000. Here, the drive module of the surgical instrument 1000 may refer to a part or module of the surgical instrument 1000 that includes the end tool 1100, and the power transmission part 1300, the connection part 1400, and a power generation part 1500 to be described later.

As a specific example, the drive module of the surgical instrument 1000 may be a separate component mountable to the manipulation part 1200 that can be directly manipulated by the user. For example, the drive module of the surgical instrument 1000 may be detachably formed on the manipulation part 1200. In this case, the drive module of the surgical instrument 1000 may include a module body, and the module body may include a coupling structure for coupling with the manipulation part 1200.

As described above, when the drive module of the surgical instrument 1000 is detachably formed on the manipulation part 1200, the user may easily replace the drive module of the surgical instrument as necessary.

As another example, the manipulation part 1200 may be replaced with a surgical robot. In other words, the drive module of the surgical instrument 1000 may be a separate component mountable to the surgical robot.

The surgical robot may refer to a robot that may perform surgery or other procedures by being manipulated by the user (e.g., a surgeon).

As a specific example, the surgical robot may include a master robot and a slave robot.

The master robot may include manipulation members that the user can grip and manipulate with both hands, and a display member that displays images captured through a laparoscope.

The slave robot may include one or more robot arm units. Here, each of the robot arm units may be provided in the form of a module that can operate independently of each other. Here, the drive module of the surgical instrument 1000 may be mounted to each of two or more of the robot arm units. For example, the drive module of the surgical instrument 1000 may be detachably formed on the surgical robot (specifically, on the slave robot). In this case, the drive module of the surgical instrument 1000 may include a module body, and the module body may include a coupling structure for coupling with the surgical robot.

As described above, when the drive module of the surgical instrument 1000 is detachably formed on the surgical robot, the user may easily replace the drive module of the surgical instrument 1000 as necessary.

Hereinafter, for convenience of description, the technical idea of the present disclosure will be described in detail by taking the surgical instrument 1000 including the manipulation part 1200 as an example. However, those skilled in the art will understand that the surgical instrument 1000 can also be implemented as the drive module configured to be mountable to the surgical robot or the manipulation part that may be directly manipulated by a user.

The power transmission part 1300 may be formed on another end portion of the connection part 1400 and may serve to transmit power generated by the power generation part 1500 to be described later to the end tool 1100. For example, the power transmission part 1300 may be positioned between the end tool 1100 and the manipulation part 1200. As will be described later, when a user such as a medical doctor manipulates the manipulation part 1200, the power generation part 1500 generates power to control the end tool 1100, and the generated power may be transmitted to the end tool 1100 through the power transmission part 1300. The power transmission part 1300 may include a plurality of wires, a plurality of pulleys, a plurality of links, a plurality of joints, a plurality of gears, and the like.

The connection part 1400 includes a shaft 1410, which is hollow and in which one or more wires and electric wires may be accommodated. The connection part 1400 has one end portion to which the end tool 1100 is coupled and another end portion to which the power transmission part 1300 is coupled, and the power transmission part 1300 may be connected to the manipulation part 1200. That is, it may be said that the connection part 1400 may serve to connect the manipulation part 1200 to the end tool 1100.

In some embodiments, a connector (not shown) may be formed on the manipulation part 1200. The connector (not shown) may be connected to an external power source (not shown), and the connector (not shown) may also be connected to the end tool 1100 via an electric wire, and may transmit, to the end tool 1100, electrical energy supplied from the external power source (not shown). Further, the electrical energy, which is transmitted to the end tool 1100 as described above, may provide a driving force for performing a yaw rotation motion, a pitch rotation motion, an actuation motion, a staple motion, and the like of the end tool 1100 to be described later. Alternatively, the electrical energy transmitted to the end tool 1100 may provide a driving force for performing cutting and cauterizing functions of the end tool 1100, using a monopolar/bipolar or ultrasonic blade. Further, the electrical energy may also be supplied to drive the power transmission part 1300. Of course, a built-in battery may be used.

The manipulation part 1200 may include a housing 1201 forming an outer shape of the manipulation part 1200. As will be described later, at least a portion of the power generation part configured to generate power to control the end tool 1100 may be accommodated inside the housing 1201. Further, a circuit unit for controlling the operation of the power generation part and a slip ring for supplying electrical energy to the power generation part, connecting communication, or transmitting various other signals may be accommodated inside the housing.

A handle 1202 may be formed on the manipulation part 1200. The handle 1202 is a part for a user to grip. Thus, the user can use the surgical instrument 1000 according to the present disclosure while gripping the handle 1202 of the manipulation part 1200.

For example, although not shown in the drawings, a button, a switch, a lever, and the like for controlling various motions of the end tool 1100 may be further formed in the manipulation part 1200.

Hereinafter, the end tool 1100, the manipulation part 1200, the power transmission part 1300, the power generation part 1500, and the like of the surgical instrument 1000 of FIGS. 1 and 2 will be described in more detail.

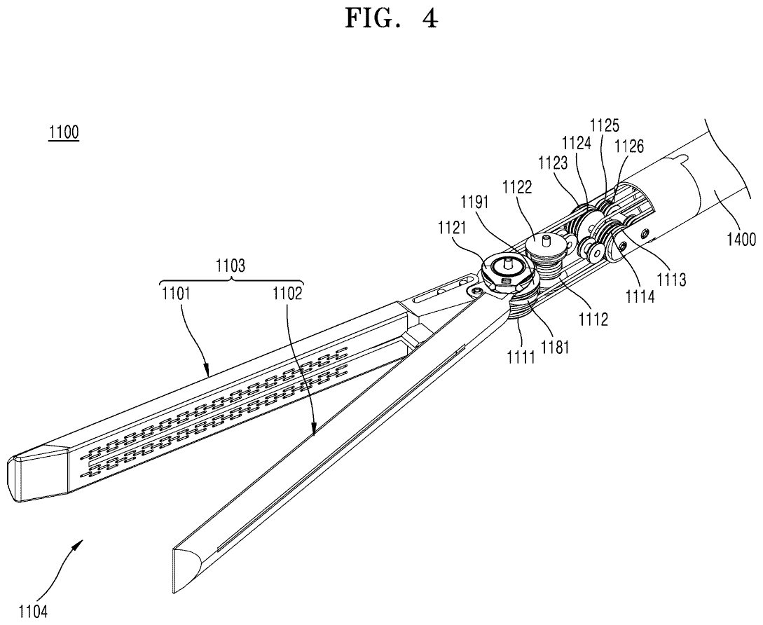

FIG. 3 is a perspective view illustrating the end tool 1100 according to an embodiment of the present disclosure, and FIG. 4 is a perspective view illustrating the end tool 1100 of FIG. 3 with an end tool hub 1106 and a pitch hub 1107 removed.

Referring to FIGS. 3 and 4, the end tool 1100 of the surgical instrument 1000 according to an embodiment of the present disclosure may include a first jaw 1101 and a second jaw 1102, each configured to be rotatable.

In other words, the end tool 1100 of the surgical instrument according to an embodiment of the present disclosure may include a pair of jaws for performing a grip motion, that is, the first jaw 1101 and the second jaw 1102. Here, each of the first jaw 1101 and the second jaw 1102, or a component encompassing the first jaw 1101 and the second jaw 1102 may be referred to as the jaw 1103.

Here, the end tool 1100 of the surgical instrument 1000 according to an embodiment of the present disclosure is configured to be rotatable in at least one direction, for example, the end tool 1100 may perform a pitch motion around a Y-axis (refer to FIG. 3) and simultaneously perform a yaw motion and an actuation motion around a Z-axis (refer to FIG. 3).

The end tool 1100 may include a plurality of pulleys including a pulley 1111 related to a rotational motion of the first jaw 1101. Further, the end tool 1100 may include a plurality of pulleys, including a pulley 1121 associated with rotational movement of the second jaw 1102.

Here, the pulleys facing each other are illustrated in the drawings as being formed parallel to each other, but the concept of the present disclosure is not limited thereto, and each of the pulleys may be variously formed with a position and a size suitable for the configuration of the end tool 1100.

Further, the end tool 1100 of the present embodiment may include the end tool hub 1106 and the pitch hub 1107.

A rotation shaft 1141 and a rotation shaft 1142 may be inserted through the end tool hub 1106, and the end tool hub 1106 may internally accommodate at least some of one or more pulleys that are axially coupled to the rotation shaft 1141. Further, the end tool hub 1106 may internally accommodate at least some of one or more pulleys that are axially coupled to the rotation shaft 1142.

For example, a pulley 1131 serving as an end tool pitch pulley may be formed at one end portion of the end tool hub 1106. Alternatively, the pulley 1131 may be integrally formed with the end tool hub 1106 as one body. That is, a disk-shaped pulley is formed at one end portion of the end tool hub 1106, and a groove around which a wire may be wound may be formed on an outer circumferential surface of the pulley. Alternatively, the pulley 1131 may be formed as a separate member from the end tool hub 1106 to be coupled to the end tool hub 1106.

A rotation shaft 1143 and a rotation shaft 1144 are inserted through the pitch hub 1107, and the pitch hub 1107 may be axially coupled to the end tool hub 1106 and the pulley 1131 by the rotation shaft 1143. Thus, the end tool hub 1106 and the pulley 1131 may be configured to be rotatable around the rotation shaft 1143 relative to the pitch hub 1107.

Further, the pitch hub 1107 may internally accommodate at least some of one or more pulleys that are axially coupled to the rotation shaft 1143. Further, the pitch hub 1107 may internally accommodate at least some of one or more pulleys that are axially coupled to the rotation shaft 1144.

Further, the end tool 1100 of the present embodiment may include the rotation shaft 1141, the rotation shaft 1142, the rotation shaft 1143, and the rotation shaft 1144. As described above, the rotation shaft 1141 and the rotation shaft 1142 may be inserted through the end tool hub 1106, and the rotation shaft 1143 and the rotation shaft 1144 may be inserted through the pitch hub 1107.

The rotation shaft 1141, the rotation shaft 1142, the rotation shaft 1143, and the rotation shaft 1144 may be arranged sequentially from a distal end 1104 of the end tool 1100 toward a proximal end 1105. Accordingly, starting from the distal end 1104, the rotation shaft 1141 may be referred to as a first pin, the rotation shaft 1142 may be referred to as a second pin, the rotation shaft 1143 may be referred to as a third pin, and the rotation shaft 1144 may be referred to as a fourth pin.

Here, the rotation shaft 1141 may function as an end tool jaw pulley rotation shaft, the rotation shaft 1142 may function as an end tool jaw auxiliary pulley rotation shaft, the rotation shaft 1143 may function as an end tool pitch rotation shaft, and the rotation shaft 1144 may function as an end tool pitch auxiliary rotation shaft of the end tool 1100.

One or more pulleys may be fitted onto each of the rotation shafts 1141 1142, 1143, and 1144.

The pulley 1111 functions as an end tool first jaw pulley, and the pulley 1121 functions as an end tool second jaw pulley. The pulley 1111 may also be referred to as a first jaw pulley, and the pulley 1121 may be referred to as a second jaw pulley, and these two components may also be referred to collectively as an end tool jaw pulley or simply a jaw pulley.

The pulley 1111 and the pulley 1121, which are end tool jaw pulleys, are formed to face each other, and are configured to be rotatable independently of each other around the rotation shaft 1141, which is an end tool jaw pulley rotation shaft. In this case, the pulley 1111 and the pulley 1121 are configured to be spaced apart from each other by a certain extent, and a staple assembly accommodation part may be formed therebetween. Further, at least some of the staple pulley assembly and the staple link assembly for stapling motion, which will be described later, may be disposed inside the staple assembly accommodation part.

Here, in the drawings, it is illustrated that the pulley 1111 and the pulley 1121 are configured to rotate around one rotation shaft 1141, but it is of course possible that each end tool jaw pulley may be configured to be rotatable around a separate shaft. Here, the first jaw 1101 is fixedly coupled to the pulley 1111 and rotated together with the pulley 1111, and the second jaw 1102 is fixedly coupled to the pulley 1121 and rotated together with the pulley 1121. Yaw rotation and actuation motions of the end tool 1100 are performed in response to the rotation of the pulley 1111 and the pulley 1121. That is, when the pulley 1111 and the pulley 1121 are rotated in the same direction around the rotation shaft 1141, the yaw rotation motion is performed, and when the pulley 1111 and the pulley 1121 are rotated in opposite directions around the rotation shaft 1141, the actuation motion is performed.

Here, the first jaw 1101 and the pulley 1111 may be formed as separate members and coupled to each other, or the first jaw 1101 and the pulley 1111 may be integrally formed as one body. Similarly, the second jaw 1102 and the pulley 1121 may be formed as separate members and coupled to each other, or the second jaw 1102 and the pulley 1121 may be integrally formed as one body.

Further, one or more auxiliary pulleys may be disposed adjacent to the pulley 1111 and the pulley 1112.

These pulleys may be formed such that one or more wires are wound therearound, the pulleys may be rotated by the wires, and the wires may move along the pulleys, thereby transmitting a driving force to the end tool 1100.

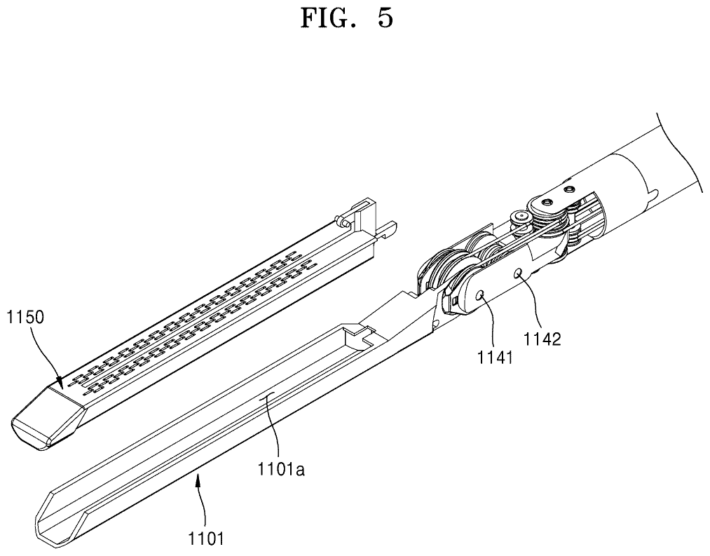

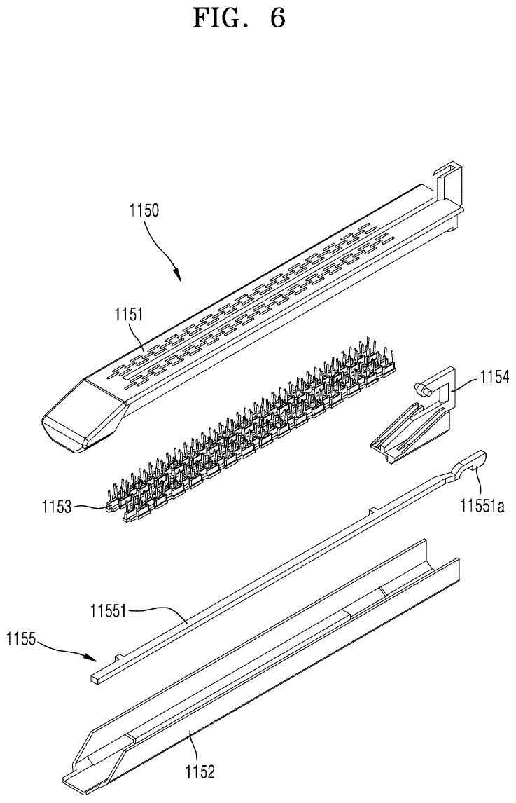

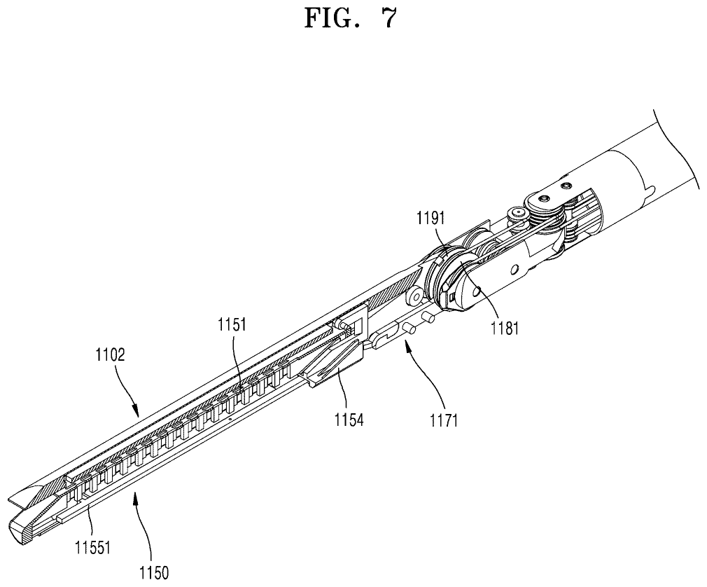

FIG. 5 is a perspective view illustrating the first jaw 1101 and a cartridge 1150 of the end tool 1100 of FIG. 3, FIG. 6 is an exploded perspective view of the cartridge of FIG. 5, and FIG. 7 is a perspective cross-sectional view for describing an internal structure of the cartridge 1150 of FIG. 6.

Referring to FIGS. 5 to 7 and the like, the cartridge 1150 is configured to be mountable to and dismountable from the first jaw 1101, and includes a plurality of staples 1153 and a blade (not shown) therein to perform suturing and cutting tissue. Here, the cartridge 1150 may include a cover 1151, a housing 1152, the staples 1153, withdrawal members (not shown), an operation member 1154, a moving member 1155, and the like.

The housing 1152 forms an outer shape of the cartridge 1150, and may be formed entirely in the form of a hollow box with one surface (upper surface) thereof removed to accommodate the moving member 1155, the operation member 1154, and the staples 1153 therein. Here, the housing 1152 may be formed in an approximately “U” shape in cross section.

The cover 1151 is formed to cover an upper portion of the housing 1152. Staple holes through which the plurality of staples 1153 may be ejected to the outside may be formed in the cover 1151. As the staples 1153, which are accommodated inside the housing 1152 before a stapling operation, are pushed and raised upward by the operation member 1154 during the stapling motion, and pass through the staple holes of the cover 1151 to be withdrawn to the outside of the cartridge 1150, stapling is performed.

In some embodiments, a slit may be formed in the cover 1151 in a longitudinal direction of the cover 1151. The blade (not shown) of the operation member 1154 may protrude out of the cartridge 1150 through the slit. As the blade (not shown) of the operation member 1154 passes along the slit, staple-completed tissue may be cut.

The plurality of staples 1153 may be positioned inside the housing 1152. As the operation member 1154, which will be described below, is linearly moved in one direction, the plurality of staples 1153 are sequentially pushed and raised from the inside of the housing 1152 to the outside, thereby performing suturing, that is, stapling. Here, the staples 1153 may be made of a material that may include titanium, stainless steel, or the like.

For example, the withdrawal members (not shown) may be further positioned between the housing 1152 and the staples 1153. In other words, it may be described that the staples 1153 are positioned above the withdrawal members (not shown). In this case, the operation member 1154 is linearly moved in one direction to push and the withdrawal members (not shown) upward, and the withdrawal members (not shown) may push the staples 1153 upward.

As such, the operation member 1154 may be described as pushing the staples 1153 upward in both the case in which the operation member 1154 directly pushes the staples 1153 upward and the case in which the operation member 1154 pushes the withdrawal members (not shown) upward and the withdrawal members (not shown) pushes the staples 1153 upward (i.e., the operation member 1154 indirectly pushes the staples 1153 upward).

The moving member 1155 may be positioned at an inner lower side of the housing 1152.

Here, the moving member 1155 is not fixedly coupled to the other components of the cartridge 1150, and may be configured to be movable relative to the other components of the cartridge 1150. That is, the moving member 1155 may perform a reciprocating linear motion relative to the housing 1152 and the cover 1151 coupled to the housing 1152.

The operation member 1154 may be positioned inside the housing 1152. The operation member 1154 is configured to be in contact with the moving member 1155, and may be configured to move linearly in one direction in response to a reciprocating linear motion of the moving member 1155. In other words, the operation member 1154 interacts with the moving member 1155 to perform stapling and cutting motions while moving in the extension direction of the connection part.

For example, the end tool 1100 according to the present embodiment may further include a first staple pulley 1181 and a second staple pulley 1191.

The first staple pulley 1181 is related to a linear motion/rotational motion of each of the pulleys and links used for stapling and cutting. The second staple pulley 1191 is related to a linear motion/rotational motion of each of the pulleys and links used for stapling and cutting. The first staple pulley 1181 and the second staple pulley 1191 are formed to respectively face the pulley 1121 and the pulley 1111, which are end tool jaw pulleys, and configured to be rotatable independently of each other around the rotation shaft 1141, which is an end tool jaw pulley rotation shaft. Here, in the drawing, it is illustrated that the first staple pulley 1181 and the second staple pulley 1191 are located between the pulley 1111 and the pulley 1121, but the concept of the present disclosure is not limited thereto, and the first staple pulley 1181 and the second staple pulley 1191 may be located at various positions adjacent to the pulley 1111 or the pulley 1121.

Here, in the present disclosure, the first staple pulley 1181, the second staple pulley 1191, the pulley 1111, and the pulley 1121 are configured to rotate around substantially the same shaft. As described above, as the first staple pulley 1181, the second staple pulley 1191, the pulley 1111, and the pulley 1121 are configured to rotate around the same shaft, it is possible to perform a pitch motion/yaw motion/actuation motion as well as stapling and cutting motions. However, here, although it is illustrated in the drawing that the first staple pulley 1181, the second staple pulley 1191, the pulley 1111, and the pulley 1121 are configured to rotate around one rotation shaft 1141, it is of course possible that each pulley may be configured to be rotatable around a separate shaft that is concentric therewith.

In other words, it may be expressed that a structure is provided in which the pulley 1111, which is a first jaw pulley, the first staple pulley 1181, the second staple pulley 1191, and the pulley 1121, which is a second jaw pulley, are sequentially stacked along the rotation shaft 1141.

Alternatively, it may be expressed that a structure is provided in which the first staple pulley 1181 and the second staple pulley 1191 are positioned between the pulley 1111 and the pulley 1121 facing each other. Here, the pulley 1111, which is a first jaw pulley, the first staple pulley 1181, the second staple pulley 1191, and the pulley 1121, which is a second jaw pulley, may be configured to be rotatable independently of each other.

For example, the end tool 1100 described above is one example of the end tool that may be mounted to the surgical instrument 1000 according to the present disclosure, and the technical concept of the present disclosure is not limited thereto and some components may be changed, omitted or added as needed.

As described above, the end tool 1100 according to an embodiment of the present disclosure may perform a yaw motion/pitch motion/actuation motion as the wire transmits power by moving along the pulleys. Further, in an embodiment of the present disclosure, the end tool 1100 may perform a firing motion by transmitting power through the movement of the wire along the pulleys, thereby enabling the linear movement of the operation member 1154 of the cartridge 1150.

That is, the wire transmits power in performing the operations of the end tool 1100. In the above, the principle of power transmission of the surgical instrument 1000 of the present disclosure will be described in detail below.

Next, the driving of the surgical instrument 1000 will be described with a focus on the configuration of the power generation part 1500.

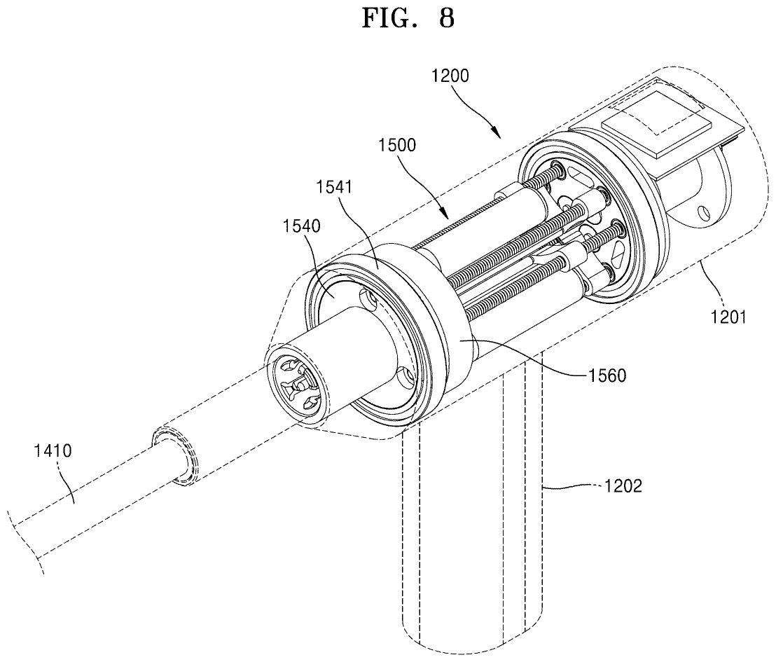

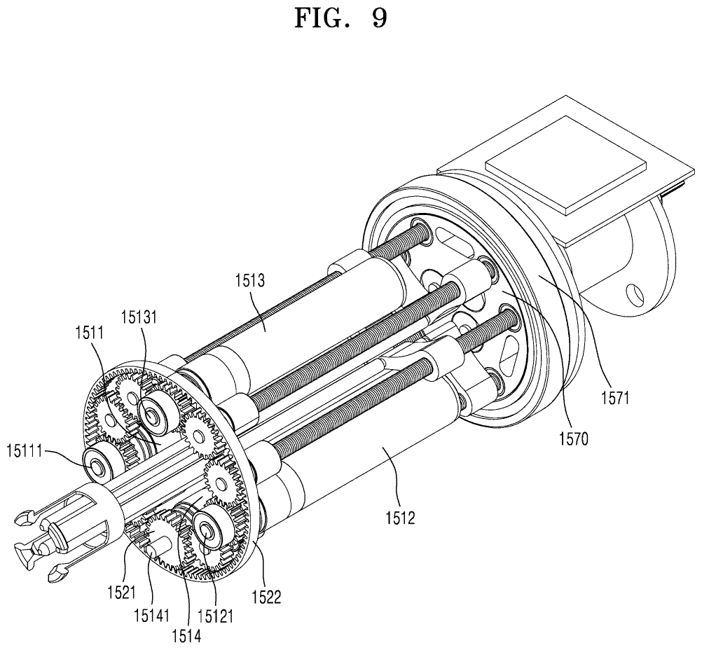

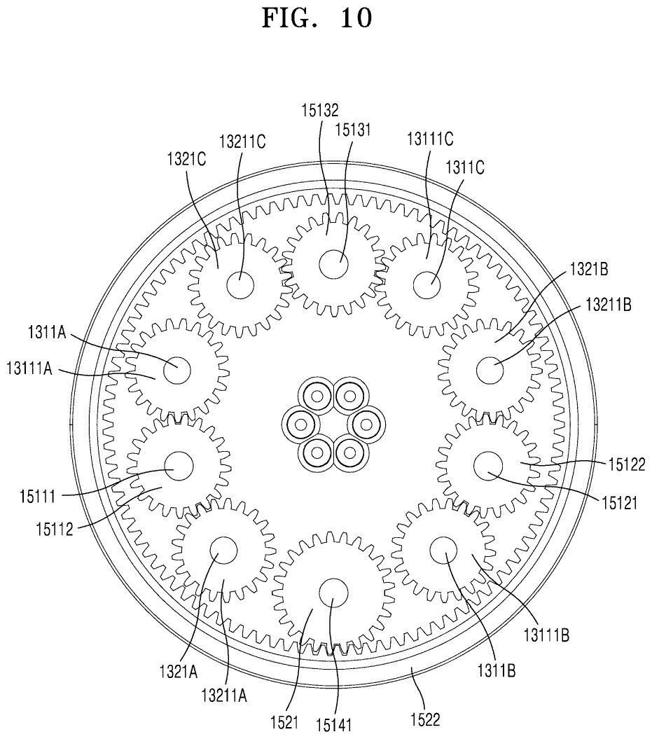

FIG. 8 is a perspective view illustrating the manipulation part 1200 and the power generation part 1500 according to an embodiment of the present disclosure, and FIG. 9 is a magnified perspective view illustrating the power generation part 1500 of FIG. 8. FIG. 10 is a cross-sectional view for describing a gear structure of the power generation part 1500 of FIG. 8.

Referring to FIGS. 8 to 10, the surgical instrument 1000 according to an embodiment of the present disclosure may include the power generation part 1500 configured to generate power to control the end tool 1100.

The power generation part 1500 may be disposed to be at least partially accommodated in the housing 1201 of the manipulation part 1200. Here, the housing 1201 may refer to a component that forms an outer shape of the manipulation part 1200, but in the case of the drive module for the surgical instrument, the housing 1201 may refer to a component that forms an outer shape of the module body.

When a user manipulates the manipulation part 1200, the power generation part 1500 may generate power to control the end tool 1100 based on the manipulation.

The power generation part 1500 may include a motor pack 1510 including at least one motor.

The motor pack 1510 may include at least one motor. For example, the motor pack 1510 may include at least one motor that generates power to drive the end tool 1100 based on a signal input to the manipulation part 1200.

The motor pack 1510 may include a yaw drive motor 1511. The yaw drive motor 1511 may generate power to yaw-rotate the end tool 1100. For example, the yaw drive motor 1511 may generate a driving force for yaw rotation of the end tool 1100 when a user manipulates the manipulation part 1200 to yaw-rotate the end tool 1100.

The driving force generated by the yaw drive motor 1511 may be transmitted to the power transmission part 1300 to move yaw drive wires 1361 and 1362, thereby allowing the end tool 1100 to yaw-rotate.

The yaw drive motor 1511 may include a yaw motor rotation shaft 15111 formed to extend in one direction. The yaw motor rotation shaft 15111 is a part that rotates when the yaw drive motor 1511 starts driving. For example, the yaw motor rotation shaft 15111 may be formed to extend in a direction toward the power transmission part 1300 from a body of the yaw drive motor 1511. As the yaw motor rotation shaft 15111 rotates, the yaw drive wires 1361 and 1362 may move, and as a result, the end tool 1100 may perform a yaw rotation or an actuation motion.

The motor pack 1510 may include a pitch drive motor 1512. The pitch drive motor 1512 may generate power to pitch-rotate the end tool 1100. For example, the pitch drive motor 1512 may generate a driving force for pitch rotation of the end tool 1100 when a user manipulates the manipulation part 1200 to pitch-rotate the end tool 1100.

The driving force generated by the pitch drive motor 1512 may be transmitted to the power transmission part 1300 to move pitch drive wires 1363 and 1364, thereby enabling the pitch rotation of the end tool 1100.

The pitch drive motor 1512 may include a pitch motor rotation shaft 15121 formed to extend in one direction. The pitch motor rotation shaft 15121 is a part that rotates when the pitch drive motor 1512 starts driving. For example, the pitch motor rotation shaft 15121 may be formed to extend in a direction toward the power transmission part 1300 from a body of the pitch drive motor 1512. As the pitch motor rotation shaft 15121 rotates, the pitch drive wires 1363 and 1364 may move, and as a result, the end tool 1100 may pitch-rotate.

The motor pack 1510 may include a firing drive motor 1513. The firing drive motor 1513 may generate power to linearly move the operation member 1154 provided in the end tool 1100. For example, the firing drive motor 1513 may generate a driving force to linearly move the operation member 1154 when a user manipulates the manipulation part 1200 to linearly move the operation member 1154 of the end tool 1100.

The driving force generated by the firing drive motor 1513 may be transmitted to the power transmission part 1300 to move firing drive wires 1365 and 1366, thereby enabling the operation member 1154 to move in the cartridge 1150 of the end tool 1100.

The firing drive motor 1513 may include a firing motor rotation shaft 15131 formed to extend in one direction. The firing motor rotation shaft 15131 is a part that rotates when the firing drive motor 1513 is driven. For example, the firing motor rotation shaft 15131 may be formed to extend in a direction toward the power transmission part 1300 from a body of the firing drive motor 1513. As the firing motor rotation shaft 15131 rotates, the firing drive wires 1365 and 1366 may move, and as a result, in the end tool 1100, the operation member 1154 linearly moves to perform stapling and cutting motions.

The power generation part 1500 may include a roll drive motor 1514 configured to generate power to roll-rotate the motor pack 1510. For example, the roll drive motor 1514 may generate a driving force to roll-rotate the motor pack 1510 when a user manipulates the manipulation part 1200 to rotate the motor pack 1510.

In an embodiment, the roll drive motor 1514 may be included in the motor pack 1510. For example, the roll drive motor 1514 may be provided inside the motor pack 1510 together with the yaw drive motor 1511 and the pitch drive motor 1512.

The roll drive motor 1514 may be driven by the user's manipulation to generate a driving force to rotate the motor pack 1510. In this case, the roll drive motor 1514 may move together with the motor pack 1510. For example, when the motor pack 1510 is rotated by the roll drive motor 1514, the roll drive motor 1514, as a component provided in the motor pack 1510, may rotate together with the motor pack 1510.

In another embodiment, the roll drive motor 1514 may not be provided in the motor pack 1510. For example, the roll drive motor 1514 may be positioned outside the motor pack 1510.

The roll drive motor 1514 may be driven by the user's manipulation to generate a driving force to rotate the motor pack 1510. In this case, the roll drive motor 1514 may move independently from the motor pack 1510. For example, unlike what will be described below, the motor pack 1510 may perform a roll rotation by the roll drive motor 1514, but the roll drive motor 1514 may not rotate together with the motor pack 1510. Specifically, the yaw drive motor 1511, the pitch drive motor 1512, and the firing drive motor 1513 may rotate together, whereas the roll drive motor 1514 may not rotate together therewith.

Hereinafter, for convenience of description, the roll drive motor 1514 will be described as being included in the motor pack 1510 by way of example, but it will be understood by those skilled in the art that the roll drive motor 1514 may not be included in the motor pack 1510 in the following description. In addition, those skilled in the art will understand that, when the roll drive motor 1514 is not provided in the motor pack 1510, the following content may be appropriately modified and applied.

The roll drive motor 1514, along with a roll drive gear 1521 and a rotation guide unit 1522, which will be described later, may serve as a roll rotation unit 1520 to roll-rotate the motor pack 1510. The principle by which the motor pack 1510 roll-rotates will be described in detail below.

The motor pack 1510 may include a base plate 1560. The base plate 1560 may be positioned in front of the yaw drive motor 1511, the pitch drive motor 1512, the roll drive motor 1514, and the firing drive motor 1513. The base plate 1560 may be connected to the yaw drive motor 1511, the pitch drive motor 1512, the roll drive motor 1514, and the firing drive motor 1513. In other words, it may be said that the roll drive motor 1514, the yaw drive motor 1511, the pitch drive motor 1512, and the firing drive motor 1513 are connected to the base plate 1560. In other words, the base plate 1560 may connect the yaw drive motor 1511, the pitch drive motor 1512, the roll drive motor 1514, and the firing drive motor 1513 to each other such that the yaw drive motor 1511, the pitch drive motor 1512, the roll drive motor 1514, and the firing drive motor 1513 move or rotate together as one body.

Thus, when the base plate 1560 rotates due to the driving force of the roll drive motor 1514, the yaw drive motor 1511, the pitch drive motor 1512, the roll drive motor 1514, and the firing drive motor 1513 connected to the base plate 1560 may rotate simultaneously. That is, when the roll drive motor 1514 is driven, the base plate 1560 is rotated, and when the base plate 1560 rotates, the yaw drive motor 1511, the pitch drive motor 1512, the roll drive motor 1514, and the firing drive motor 1513 connected to the base plate 1560 are rotated together with the base plate 1560. Here, since the base plate 1560 rotates around an axis formed in a direction in which the connection part 1400 extends, the motor pack 1510 including the base plate 1560, the yaw drive motor 1511, the pitch drive motor 1512, the roll drive motor 1514, and the firing drive motor 1513 roll-rotates around the axis formed in the direction in which the connection part 1400 extends.

In an embodiment, the yaw drive motor 1511, the pitch drive motor 1512, the roll drive motor 1514, the firing drive motor 1513 may be disposed side by side with each other. Further, the yaw drive motor 1511, the pitch drive motor 1512, the roll drive motor 1514, and the firing drive motor 1513 may be positioned to form a circular pattern.

As described above, the motor pack 1510 may roll-rotate inside the housing 1201 of the manipulation part 1200. In this case, since the motor pack 1510 includes a plurality of motors, by positioning the yaw drive motor 1511, the pitch drive motor 1512, the roll drive motor 1514, and the firing drive motor 1513 to form a circular pattern with each other, when the motor pack 1510 rotates, a diameter of a space occupied by the rotation of the motor pack 1510 may be minimized. That is, by positioning the yaw drive motor 1511, the pitch drive motor 1512, the roll drive motor 1514, and the firing drive motor 1513 to form a circular pattern, an inner diameter of the housing 1201 necessary for the rotation of the motor pack 1510 may be designed to be small, which may contribute to miniaturization and lightweighting of the surgical instrument 1000.

In some embodiments, here, positioning the yaw drive motor 1511, the pitch drive motor 1512, the firing drive motor 1513, and the roll drive motor 1514, to form a circular pattern does not imply equal spacing therebetween. Instead, it is sufficient when outer circumferential surfaces of the yaw drive motor 1511, the pitch drive motor 1512, the firing drive motor 1513, and the roll drive motor 1514 are positioned within a circle.

In an embodiment, the yaw drive motor 1511, the pitch drive motor 1512, firing drive motor 1513, and roll drive motor 1514 may be provided with different performances from one another. For example, the magnitudes of driving forces that the yaw drive motor 1511, the pitch drive motor 1512, the firing drive motor 1513, and the roll drive motor 1514 must generate to perform their respective roles may be different from each other. To this end, the yaw drive motor 1511, the pitch drive motor 1512, the firing drive motor 1513, and the roll drive motor 1514 may have different outputs or sizes as necessary.

At least one through hole may be formed in the base plate 1560. For example, at least as many through holes as the number of motors included in the motor pack 1510 may be formed in the base plate 1560. The through hole is a part through which the rotation shaft of the respective motor passes.

For example, the yaw motor rotation shaft 15111 extends from the body of the yaw drive motor 1511, and may be formed to extend through the base plate 1560. In addition, the pitch motor rotation shaft 15121 extends from the body of the pitch drive motor 1512, and may be formed to extend through the base plate 1560. Further, the firing motor rotation shaft 15131 extends from the body of the firing drive motor 1513, and may extend through the base plate 1560. Further, a roll motor rotation shaft 15141 extends from a body of the roll drive motor 1514, and may be formed to extend through the base plate 1560.

The principle by which the motor pack 1510 roll-rotates in the surgical instrument 1000 of the present disclosure will be described below.

The motor pack 1510 may roll-rotate in the direction in which the connection part 1400 extends.

Here, a roll motion as used herein is defined as follows.

The roll motion refers to a motion in which the end tool 1100, the connection part 1400, the motor pack 1510, and the like, which constitute the surgical instrument 1000, rotate around an axis formed in the direction in which the connection part 1400 extends. In other words, the roll motion refers to a motion of rotating around an axis formed in an extension direction (the X-axis direction in FIG. 3) of the connection part 1400 without bending in a Y-axis direction of FIG. 3 or a Z-axis direction of FIG. 3.

At least a portion of the power generation part 1500 may be accommodated inside the housing 1201 of the manipulation part 1200. The power generation part 1500 may include the motor pack 1510 and a motor housing forming the exterior of the motor pack 1510. The motor housing may be integrally formed with the housing 1201 of the manipulation part 1200 or may be provided as a separate component. For example, the following description focuses on an embodiment in which the motor housing is integrally formed with the housing 1201 of the manipulation part 1200 and accommodates the motor pack 1510 therein.

The phrase “the motor pack 1510 roll-rotates” may mean that the motor pack 1510 rotates inside the housing 1201 along an inner circumferential surface of the housing 1201. In other words, when a user performs a manipulation to roll-rotate the manipulation part 1200 while grasping the handle 1202 of the manipulation part 1200, the motor pack 1510 may rotate around the axis, which is formed in the direction in which the connection part 1400 extends, inside the housing 1201 while the housing 1201 and the handle 1202 of the manipulation part 1200 are fixed in place. In other words, it may be said that the housing 1201 and the handle 1202 rotate when the user performs a manipulation for a roll motion while grasping the connection part 1400.

The power generation part 1500 may include the roll rotation unit 1520 that roll-rotates the motor pack 1510.

The roll rotation unit 1520 may include the roll drive motor 1514, the roll drive gear 1521, and the rotation guide unit 1522.

As described above, the roll drive motor 1514 may generate a driving force to roll-rotate the motor pack 1510.

The roll drive motor 1514 may include the roll motor rotation shaft 15141 formed to extend in one direction. The roll motor rotation shaft 15141 is a part that rotates when the roll drive motor 1514 is driven. For example, the roll motor rotation shaft 15141 may be formed to extend in a forward-facing direction from the body of the roll drive motor 1514. That is, the roll motor rotation shaft 15141 may extend in a forward-facing direction from the body of the roll drive motor 1514. As the roll motor rotation shaft 15141 rotates, the motor pack 1510 may roll-rotate.

The roll drive gear 1521 may be connected to the roll drive motor 1514. As shown in FIGS. 9 and 10, the roll drive gear 1521 is positioned on one side of the roll drive motor 1514 and may rotate together with the roll drive motor 1514. That is, the roll motor rotation shaft 15141 is formed to extend forward to pass through the base plate 1560 from the body of the roll drive motor 1514, and the roll drive gear 1521 may be positioned in the extending portion of the roll motor rotation shaft 15141.

The roll drive gear 1521 may be a gear having gear teeth formed on an outer circumferential surface thereof. The roll drive gear 1521 is engaged with the rotation guide unit 1522, and may rotate the motor pack 1510 in response to the driving of the roll drive motor 1514.

The rotation guide unit 1522 is engaged with the roll drive gear 1521, and may rotate the motor pack 1510 by the driving of the roll drive motor 1514.

In an embodiment, the rotation guide unit 1522 may be a circular gear, as shown in FIG. 9. The rotation guide unit 1522 may be formed in the shape of a hollow circle, and gear teeth may be formed on an inner circumferential surface of the circle. That is, the rotation guide unit 1522 may be a type of ring gear with gear teeth formed on an inner circumferential surface thereof.

The rotation guide unit 1522 may be positioned in front of the base plate 1560. Further, the rotation guide unit 1522 may be fixed to the inner circumferential surface of the housing 1201. Accordingly, when the roll drive motor 1514 is driven, the motor pack 1510 may roll-rotate inside the housing 1201.

Specifically, when the roll drive motor 1514 is driven, the roll motor rotation shaft 15141 may rotate, and the roll drive gear 1521 positioned on the roll motor rotation shaft 15141 may rotate together with the roll motor rotation shaft 15141. When the roll drive gear 1521 rotates, the rotation guide unit 1522, which is engaged with the roll drive gear 1521, remains fixed to the inner circumferential surface of the housing 1201. As a result, the roll drive gear 1521 moves along the gear teeth of the rotation guide unit 1522. That is, when the roll drive motor 1514 starts driving, the roll drive gear 1521 and the rotation guide unit 1522 rotate relatively to each other, and since the rotation guide unit 1522 is fixed to the housing 1201, the roll drive gear 1521 moves relatively along the rotation guide unit 1522.

Further, the roll drive gear 1521 is connected to the roll motor rotation shaft 15141, the roll drive motor 1514 is connected to the base plate 1560, and the base plate 1560 is connected to the firing drive motor 1513, the yaw drive motor 1511, and the pitch drive motor 1512. Accordingly, the motor pack 1510 may rotate relative to the housing 1201 by the operation of the roll drive gear 1521. In other words, the motor pack 1510 may rotate independently of the movement of the housing 1201.

More specifically, the base plate 1560 may rotate relative to the housing 1201. That is, since the roll drive gear 1521 is connected to the base plate 1560 by the roll motor rotation shaft 15141, the base plate 1560 may rotate relative to the housing 1201 as the roll drive gear 1521 moves along the rotation guide unit 1522. Here, since the roll motor rotation shaft 15141 is eccentric with respect to the rotation shaft of the base plate 1560, when the roll drive gear 1521 moves along the rotation guide unit 1522, the base plate 1560 may rotate relative to the housing 1201, rather than change in position along the roll drive gear 1521.

In the drawings, the roll drive gear 1521 and the rotation guide unit 1522 are illustrated in the form of spur gears, but the present disclosure is not limited thereto, and it is of course possible that the roll drive gear 1521 and the rotation guide unit 1522 may have various other gear forms, such as helical gears and herringbone gears. Further, when the roll drive motor 1514 is positioned outside the motor pack 1510, the positions of the roll drive gear 1521 and the rotation guide unit 1522 may vary.

In an embodiment, the power generation part 1500 may further include a bearing plate 1540 and a first bearing 1541. The bearing plate 1540 and the first bearing 1541 may reduce rotational friction between the motor pack 1510 and the housing 1201 when the motor pack 1510 roll-rotates.

The bearing plate 1540 may be positioned in front of the rotation guide unit 1522.

At least one or more through holes may be formed in the bearing plate 1540. The through holes are parts through which the rotation shaft of the respective motor passes.

For example, the yaw motor rotation shaft 15111 may be formed to extend from the body of the yaw drive motor 1511 to pass through the base plate 1560 and the bearing plate 1540. Further, the pitch motor rotation shaft 15121 may be formed to extend from the body of the pitch drive motor 1512 to pass through the base plate 1560 and the bearing plate 1540. Further, the firing motor rotation shaft 15131 may be formed to extend from the body of the firing drive motor 1513 to pass through the base plate 1560 and the bearing plate 1540.

For example, the roll motor rotation shaft 15141 is formed to extend from the body of the roll drive motor 1514 to pass through the base plate 1560, but may not extend to the bearing plate 1540. The bearing plate 1540, like the base plate 1560, may also be connected to the roll drive gear 1521 by the roll motor rotation shaft 15141. Thus, when the roll drive gear 1521 rotates, the bearing plate 1540 may rotate relative to the housing 1201 as the roll drive gear 1521 moves along the rotation guide unit 1522. In this case, the first bearing 1541 may be positioned to be in contact with the inner circumferential surface of the housing 1201, coaxially with the bearing plate 1540, to reduce rotational friction of the bearing plate 1540.

As such, since the yaw motor rotation shaft 15111, the pitch motor rotation shaft 15121, and the firing motor rotation shaft 15131 are formed to extend through the bearing plate 1540, when the motor pack 1510 rotates, the base plate 1560 and the bearing plate 1540 may rotate together with the motor pack 1510.

The first bearing 1541 may be positioned on an outer circumferential surface of the bearing plate 1540. For example, the first bearing 1541 may be disposed to cover the outer circumferential surface of the bearing plate 1540. Thus, when the motor pack 1510 roll-rotates, the bearing plate 1540 rotates together with the motor pack 1510, and in this case, the bearing plate 1540 and the first bearing 1541 may reduce the rotational friction between the motor pack 1510 and the housing 1201.

In an embodiment, the power generation part 1500 may further include a circuit plate 1570 and a second bearing 1571. The circuit plate 1570 and the second bearing 1571 may reduce the rotational friction between the motor pack 1510 and the housing 1201 when the motor pack 1510 roll-rotates.

The circuit plate 1570 may be positioned at the rear of the motor pack 1510. As will be described later, the circuit plate 1570 is a part to which a circuit unit 1600 is connected.

The circuit plate 1570 is connected to the motor pack 1510, and may rotate together with the motor pack 1510 in response to the rotation of the motor pack 1510. Further, a second bearing 1571 may be positioned on an outer circumferential surface of the circuit plate 1570. Thus, when the motor pack 1510 roll-rotates, the circuit plate 1570 rotates together with the motor pack 1510, and in this case, the circuit plate 1570 and the second bearing 1571 may reduce the rotational friction between the motor pack 1510 and the housing 1201.

The surgical instrument 1000 according to an embodiment of the present disclosure may further include the circuit unit 1600 for controlling the power generation part 1500.

The circuit unit 1600 may include an electronic circuit for controlling the driving of the motor pack 1510. The circuit unit 1600 may include a motor driver, a motor controller, a microcontroller unit, and the like, but is not limited thereto, and may have various other components capable of driving the motor pack 1510.

The circuit unit 1600 may be positioned in the manipulation part 1200. A manipulation-part internal space 1203 may be provided inside the manipulation part 1200, and the circuit unit 1600 may be positioned in the manipulation-part internal space 1203.

The circuit unit 1600 may be positioned on one side of the motor pack 1510. For example, the circuit unit 1600 may be positioned at the rear of the motor pack 1510, i.e., in a direction opposite to the connection part 1400. In more detail, the circuit unit 1600 may be connected to the circuit plate 1570 of the power generation part 1500. Thus, when the motor pack 1510 roll-rotates, the circuit unit 1600 may rotate together with the motor pack 1510.

Although not shown in the drawings, in order to drive the motor pack 1510, the circuit unit 1600 and the motor pack 1510 may be connected to each other through a plurality of electric wires. Thus, as the motor pack 1510 roll-rotates, the circuit unit 1600 also rotates together with the motor pack 1510, so that an issue of twisting the plurality of electric wires connecting the motor pack 1510 to the circuit unit 1600 can be prevented.

In an embodiment, a slip ring (not shown) may be positioned on one side of the circuit unit 1600. The slip ring is a component for connecting various electrical/electronic elements or establishing communication with the circuit unit 1600 that controls the driving of the motor pack 1510 or the motor pack 1510. For example, the slip ring may electrically connect the circuit unit, which controls the driving of the motor pack 1510 or the motor pack 1510, to a power supply, a switch, a button, an organic light-emitting diode (OLED) screen, and other circuit units. Here, the power supply, the switch, the button, the OLED screen, and other circuit units may be positioned inside the surgical instrument 1000 according to the present disclosure or may be positioned outside the surgical instrument 1000. Further, the slip ring may establish communication between various elements for the operation of the surgical instrument. For example, the slip ring may establish communication between at least some of the manipulation part, the power transmission part, the power generation part, and the circuit unit. In this case, the type of communication is not limited, and any type that can communicatively connect the various elements of the surgical instrument 1000 may be employed.

As described above, the motor pack 1510 and the circuit unit 1600 are configured to be roll-rotatable. Further, the motor pack 1510 and/or the circuit unit 1600 may be electrically connected to various electrical/electronic elements. In this case, when the motor pack 1510 and/or the circuit unit 1600 are connected to the electrical/electronic elements through electric wires or the like, the electric wires may be twisted by the rotation of the motor pack 1510 and the circuit unit 1600.

Thus, by positioning the slip ring on one side of the circuit unit 1600, even when the circuit unit 1600 rotates, the electric wires connecting various electrical/electronic elements to the motor pack 1510 and/or the circuit unit 1600 may not be twisted. For example, since the electric wires are not twisted, the motor pack 1510 and the circuit unit 1600 may stably receive power from an external power supply.

In an embodiment, although not shown in the drawings, the surgical instrument may further include at least one sub-circuit unit. The sub-circuit unit may be disposed inside the manipulation part 1200. In an embodiment, the sub-circuit unit may be positioned on a portion of the handle 1202 of the manipulation part 1200. In this case, the sub-circuit unit may not rotate even when the motor pack 1510 rotates.

The sub-circuit unit may preprocess various signals for controlling the motor pack 1510. Further, the sub-circuit unit may transmit the preprocessed signals to the circuit unit 1600. To this end, the circuit unit 1600 may be connected to the sub-circuit unit by serial communication or the like, but the present disclosure is not limited thereto, and the circuit unit 1600 may be connected to the sub-circuit unit in various ways. Thus, the number of electric wires that need to be connected to the circuit unit 1600 may be reduced by using the slip ring.

As a specific example, when it is assumed that the manipulation part 1200 has four buttons for manipulation, and each button requires two electric wires (e.g., one electric wire for grounding and another electric wire for communication) to send and receive signals, at least five electric wires should be connected to the circuit unit 1600. That is, even when the electric wire for grounding is commonly used, at least one electric wire for grounding and four electric wires for communication are required for the manipulating electric wires in the manipulation part 1200. In this case, when the sub-circuit unit is included as in the present embodiment, and preprocesses signals from at least five electric wires simultaneously, through the communication connection function of the slip ring, the four buttons of the circuit unit 1600 can be communicated with the manipulation part 1200 using only two electric wires. Thus, this configuration can simplify the arrangement of electric wires and further minimize the size of the slip ring. However, this example represents just one of various functions of the sub-circuit unit, and in addition thereto, the sub-circuit unit can preprocess signals of various electric wires, and thus, the technical contents of the present disclosure are not limited to the description provided above.

In an embodiment, although not shown in the drawings, the surgical instrument 1000 may further include a component for setting a zero point of roll rotation of the motor pack 1510 or the like. For example, the surgical instrument 1000 may further include at least one encoder for measuring a roll rotation angle of the motor pack 1510 or the like. Alternatively, the surgical instrument 1000 may further include a touch sensor, a Hall effect sensor, a photo sensor, or the like to measure the roll rotation angle of the motor pack 1510. However, the present disclosure is not limited thereto, and any component capable of measuring the roll rotation angle of the motor pack 1510 or the like may be provided in the surgical instrument 1000 of the present disclosure.

In the above, the principle of power generation of the surgical instrument 1000 according to an embodiment of the present disclosure and the driving of the motor pack 1510 have been described. For example, as described above, when power is generated by the power generation part 1500 of the surgical instrument 1000 of the present disclosure, the wire may move along the pulleys and transmit the power to the end tool 1100. Accordingly, the end tool 1100 may perform a yaw motion, a pitch motion, an actuation motion, and a firing motion. Hereinafter, the principle of power transmission of the surgical instrument 1000 will be described in detail, with a focus on the configuration of the power transmission part 1300.

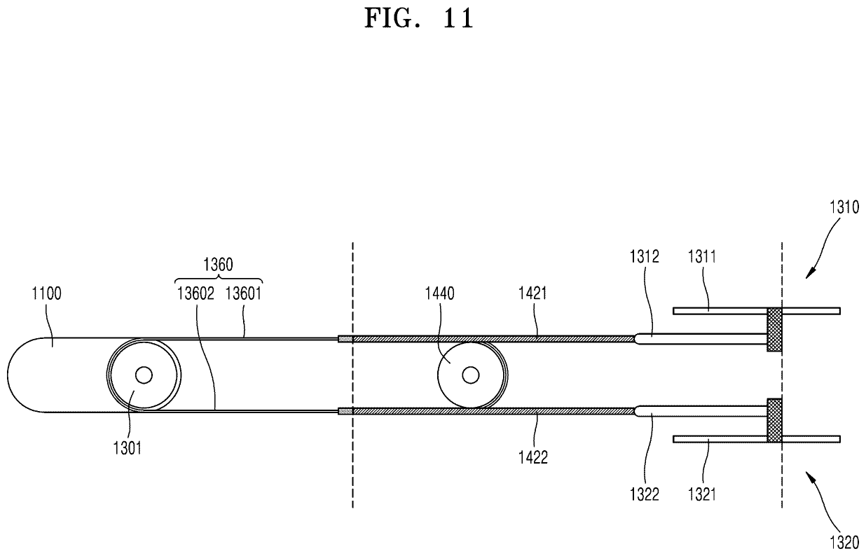

FIGS. 11 to 13 are conceptual diagrams illustrating a power transmission structure of the surgical instrument 1000 according to an embodiment of the present disclosure and operations of the end tool 1100.

Referring to FIGS. 11 to 13, the surgical instrument 1000 may include a drive wire 1360.

The drive wire 1360 may control the rotation of the end tool 1100. The drive wire 1360 connects the end tool 1100 to the power generation part 1500, enabling the power generated by the power generation part 1500 to be transmitted to the end tool 1100.

In the present specification, the drive wire 1360 may refer to all wires connected to the end tool 1100 that are capable of controlling the operation of the end tool 1100. For example, the drive wire 1360 may refer to the yaw drive wires 1361 and 1362, which move by the driving of the yaw drive motor 1511 and enable yaw rotation of the end tool 1100. Alternatively, the drive wire 1360 may refer to the pitch drive wires 1363 and 1364, which move by the driving of the pitch drive motor 1512 and enable pitch rotation of the end tool 1100. Alternatively, the drive wire 1360 may refer to the firing drive wires 1365 and 1366, which move by the driving of the firing drive motor 1513 and are capable of moving the operation member 1154 of the end tool 1100.

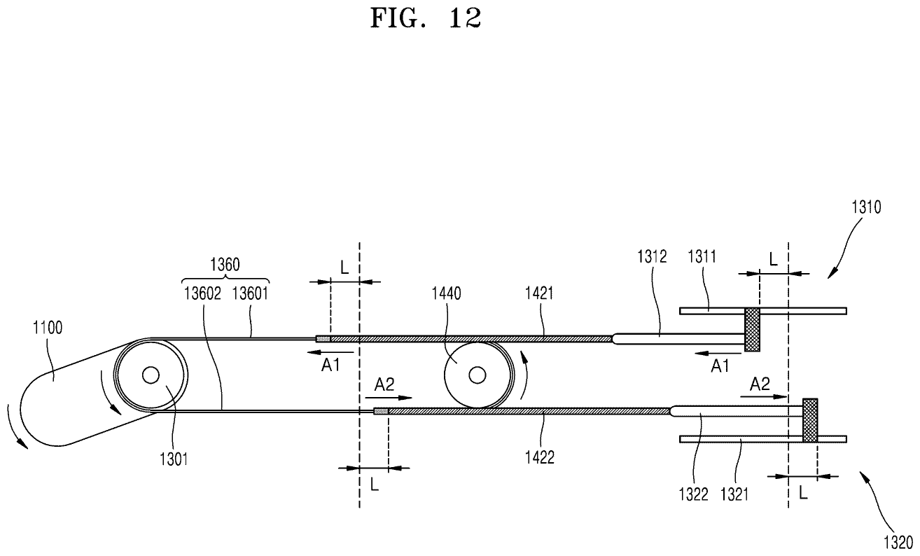

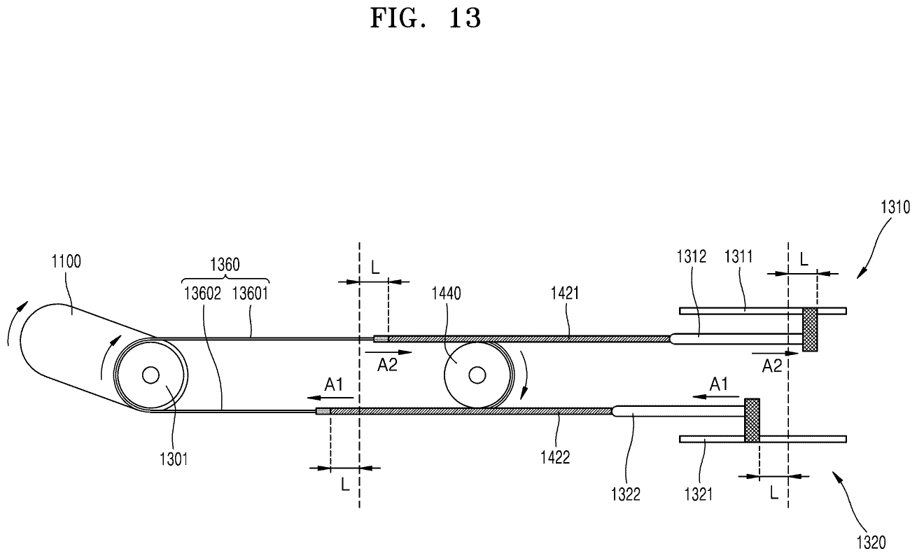

FIGS. 12 and 13 illustrate operational states in which the end tool 1100 rotates in a specific direction, but, the operation of the end tool 1100, based on power transmission in the surgical instrument 1000, is not limited thereto and may also include a linear movement of the operation member 1154, which may be controlled by the movement of the drive wire 1360.

The drive wire 1360 may have a first wire 13601 and a second wire 13602. As described above, the yaw drive wires 1361 and 1362, the pitch drive wires 1363 and 1364, and the firing drive wires 1365 and 1366 may each be provided as a pair. That is, the drive wire 1360 may have a pair of wires, which are the first wire 13601 and the second wire 13602, and the pair of wires may move to control the operation of the end tool 1100.

The first wire 13601 and the second wire 13602 may be provided as a single wire. Both strands of the drive wire 1360 centered on an intermediate point of the drive wire 1360, which is a single wire, may be referred to as the first wire 13601 and the second wire 13602, respectively. Alternatively, the first wire 13601 and the second wire 13602 may also be formed as separate wires and connected by a fastening member (not shown).

The drive wire 1360 may move as the first wire 13601 and the second wire 13602, which are wound around the driving pulley 1301, are pulled and released. The driving pulley 1301 may guide the movement of the first wire 13601 and the second wire 13602. Further, the power transmission part 1300 of the present disclosure may include one or more sub-driving pulleys (not shown) to control the movement path and direction of the drive wire 1360.

Specifically, as shown in FIG. 12, as the first wire 13601 moves in one direction (an arrow A1 direction) and the second wire 13602 moves in another direction (an arrow A2 direction), the end tool 1100 may rotate counterclockwise on the plane of the drawing. Conversely, as shown in FIG. 13, as the second wire 13602 moves in one direction (the arrow A1 direction) and the first wire 13601 moves in another direction (the arrow A2 direction), the end tool 1100 may rotate clockwise on the plane of the drawing.

That is, as one wire of the first wire 13601 and the second wire 13602 is pulled in another direction (the arrow A2 direction), the end tool 1100 may rotate toward the one wire. Accordingly, the power transmission part 1300 may control the operation direction and magnitude of the end tool 1100 by controlling the pulling direction and length of the drive wire 1360. At this time, the power transmission part 1300 may control a single operation by controlling the pair of drive wires 13601 and 13602 to move in opposite directions.

The power transmission part 1300 may include a first power transmission unit 1310 and a second power transmission unit 1320.

The first power transmission unit 1310 is connected to the first wire 13601, and may control the movement of the first wire 13601. The first power transmission unit 1310 may move linearly in the longitudinal direction by the driving of the motor and control the movement of the first wire 13601.

The first power transmission unit 1310 may have a first lead screw 1311 and a first linear movement guide 1312.

Specifically, the first power transmission unit 1310 may cause the first lead screw 1311 to rotate by the driving of the motor, thereby allowing the first linear movement guide 1312 to linearly move. A first wire coupling member 1421 of the connection part 1400 to be described later may be coupled to one end of the first wire 13601. Accordingly, as the first linear movement guide 1312 linearly moves to push the first wire coupling member 1421, the first wire coupling member 1421 also moves linearly, thereby moving the first wire 13601.

The second power transmission unit 1320 is connected to the second wire 13602 and may control the movement of the second wire 13602. The second power transmission unit 1320 may move linearly in the longitudinal direction by the driving of the motor and control the movement of the second wire 13602.

The second power transmission unit 1320 may include a second lead screw 1321 and a second linear movement guide 1322.

Specifically, the second power transmission unit 1320 may cause the second lead screw 1321 to rotate by the driving of the motor, thereby allowing the second linear movement guide 1322 to linearly move. A second wire coupling member 1422 of the connection part 1400 to be described later may be coupled to one end of the second wire 13602. Accordingly, as the second linear movement guide 1322 linearly moves to push the second wire coupling member 1422, the second wire coupling member 1422 also moves linearly while remaining in contact with the second linear movement guide 1322, thereby moving the second wire 13602.

The connection part 1400 may include the first wire coupling member 1421 and the second wire coupling member 1422.

The first wire coupling member 1421 may move linearly with one end connected to the first wire 13601 and another end remaining in contact with the first linear movement guide 1312. When the first linear movement guide 1312 moves linearly by the driving of the motor, the first wire coupling member 1421 may be pushed by the first linear movement guide 1312 and may move linearly. The first wire 13601 connected to the first wire coupling member 1421 may also move linearly in response to the movement of the first wire coupling member 1421.

The second wire coupling member 1422 may move linearly with one end connected to the second wire 13602 and another end remaining in contact with the second linear movement guide 1322. When the second linear movement guide 1322 moves linearly by the driving of the motor, the second wire coupling member 1422 may be pushed by the second linear movement guide 1322 and may move linearly. The second wire 13602 connected to the second wire coupling member 1422 may also move linearly in response to the movement of the second wire coupling member 1422.

The connection part 1400 may further include a direction-changing member 1440. The direction-changing member 1440 may switch movement directions between the first wire 13601 and the second wire 13602.

The first wire 13601 and the second wire 13602 may extend while being wound around the driving pulley 1301. At this time, one end of the first wire 13601 may be coupled to the first wire coupling member 1421, and one end of the second wire 13602 may be coupled to the second wire coupling member 1422. When the first linear movement guide 1312 moves linearly in one direction (the arrow A1 direction) while remaining in contact with the first wire coupling member 1421 by the driving of the motor, the first linear movement guide 1312 may push the first wire coupling member 1421, thereby allowing the first wire 13601 to move in one direction (the arrow A1 direction) together with the first wire coupling member 1421.

In some embodiments, since the second wire coupling member 1422 is only in face-to-face contact with the second linear movement guide 1322, the second linear movement guide 1322 can push the second wire coupling member 1422 in one direction (the arrow A1 direction) by a certain distance L, but cannot pull the second wire coupling member 1422 in another direction (the arrow A2 direction) by the same distance L. That is, when the first linear movement guide 1312 moves the first wire coupling member 1421 in one direction (the arrow A1 direction), the second wire coupling member 1422 cannot move in another direction (the arrow A2 direction) correspondingly. When the second wire 13602 fails to move in another direction (the arrow A2 direction) by the same distance L that the first wire 13601 has moved in one direction, a tension of the drive wire 1360 may become excessively high or low, which may cause the end tool 1100 to be unable to operate normally.

Similarly, when the second linear movement guide 1322 moves linearly in one direction (the arrow A1 direction) while remaining in contact with the second wire coupling member 1422, since the first wire coupling member 1421 is only in face-to-face contact with the first linear movement guide 1312, the first linear movement guide 1312 cannot pull the first wire coupling member 1421 in another direction (the arrow A2 direction) by the same distance L. When the first wire 13601 fails to move in another direction (the arrow A2 direction) by the same distance L that the second wire 13602 has moved in one direction, the tension of the drive wire 1360 may become excessively high or low, which may cause the end tool 1100 to be unable to operate normally.

Accordingly, in the surgical instrument 1000 of the present disclosure, the connection part 1400 further includes the direction-changing member 1440, allowing the first wire 13601 and the second wire 13602 to move in opposite directions by corresponding distances, thereby maintaining the tension of the drive wire 1360 and enabling the normal operation of the end tool 1100.

Specifically, as shown in FIG. 12, when the first linear movement guide 1312 moves in one direction (the arrow A1 direction) by the driving of the motor while pushing the first wire coupling member 1421, the first wire 13601 can move in one direction (the arrow A1 direction). When the first wire 13601 moves in one direction (the arrow A1 direction), the second wire 13602 may move in another direction (the arrow A2 direction) by the same distance L due to the direction-changing member 1440. As a result, the drive wire 1360 can maintain the tension while transmitting power to the end tool 1100, which allows the end tool 1100 to rotate toward the second wire 13602.

Similarly, as shown in FIG. 13, when the second linear movement guide 1322 moves in one direction (the arrow A1 direction) by the driving of the motor while pushing the second wire coupling member 1422, the second wire 13602 can move in one direction (the arrow A1 direction). When the second wire 13602 moves in one direction (the arrow A1 direction), the first wire 13601 may move in another direction (the arrow A2 direction) by the same distance L due to the direction-changing member 1440. As a result, the drive wire 1360 can maintain the tension while transmitting power to the end tool 1100, which allows the end tool 1100 to rotate toward the first wire 13601.

For example, the detailed structure of the power transmission part 1300 and the connection part 1400, as well as the principle of power transmission accordingly, will be described in detail below.

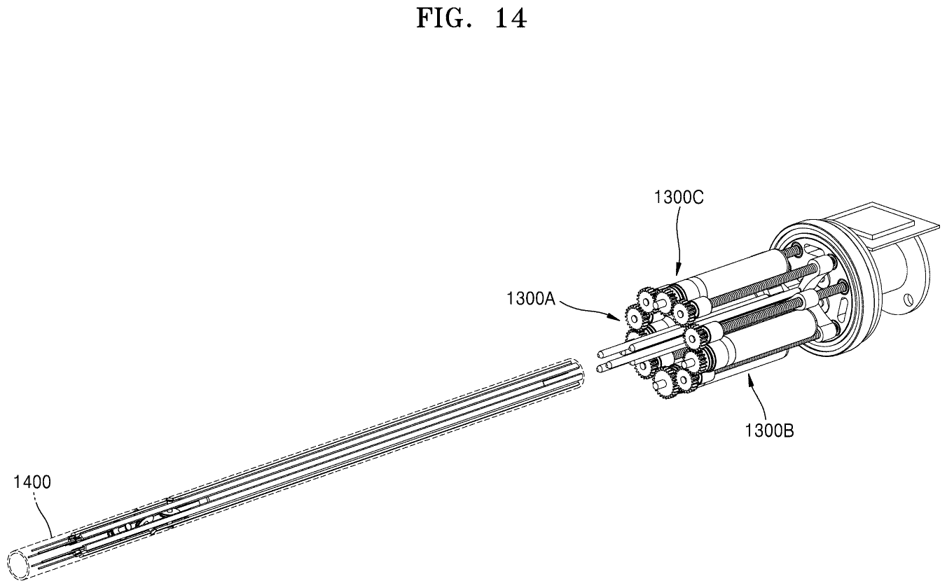

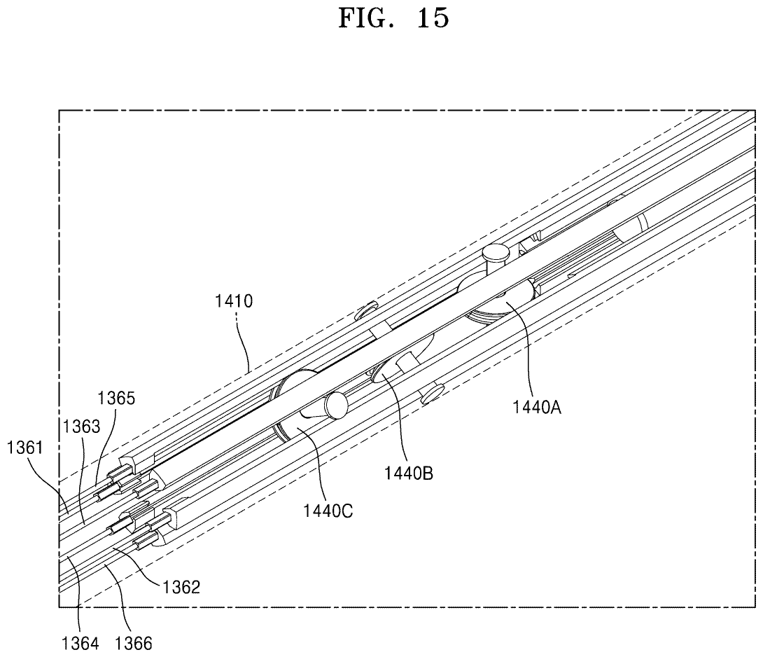

FIGS. 14 and 15 are perspective views illustrating a plurality of power transmission parts 1300 provided in the surgical instrument 1000 according to an embodiment of the present disclosure.

Referring to FIGS. 14 and 15, the surgical instrument 1000 may include the plurality of power transmission parts 1300. When the end tool 1100 of the surgical instrument 1000 performs a plurality of motions, the surgical instrument 1000 may include the plurality of power transmission parts 1300 for transmitting power to perform each motion.

For example, the end tool 1100 may perform a yaw motion, a pitch motion, an actuation motion, and a firing motion as described above. At this time, since the yaw motion and the actuation motion perform opposite operations around the same shaft, the yaw motion and the actuation motion can be practically controlled by the same motor Accordingly, the surgical instrument 1000 may include the yaw drive motor 1511 for controlling the yaw and actuation motions of the end tool 1100, the pitch drive motor 1512 for controlling the pitch motion, and the firing drive motor 1513 for controlling the firing motion. In this case, the surgical instrument 1000 may include a yaw power transmission part 1300A, a pitch power transmission part 1300B, and a firing power transmission part 1300C. That is, the yaw power transmission part 1300A may be connected to the yaw drive motor 1511, the pitch power transmission part 1300B may be connected to the pitch drive motor 1512, and the firing power transmission part 1300C may be connected to the firing drive motor 1513 to transmit power to the end tool 1100.

In the surgical instrument 1000, each power transmission part 1300 may have substantially the same detailed structure and transmit power generated by the power generation part. Hereinafter, in an embodiment in which the surgical instrument 1000 includes the yaw power transmission part 1300A, the pitch power transmission part 1300B, and the firing power transmission part 1300C, the method by which the surgical instrument 1000 transmits power will be described with a focus on the detailed structure of the yaw power transmission part 1300A.

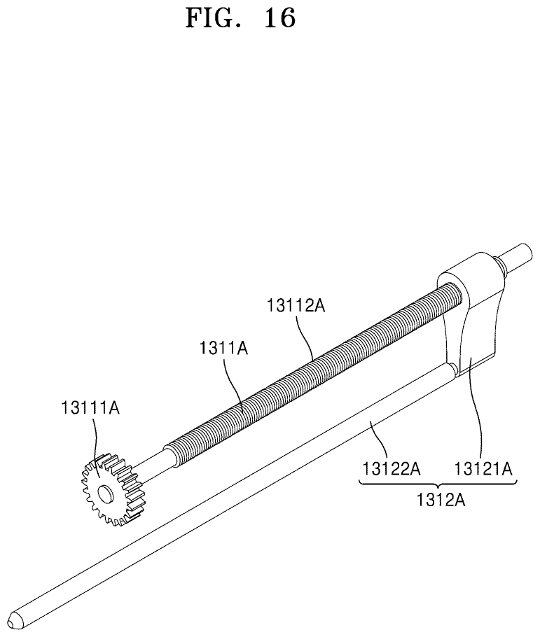

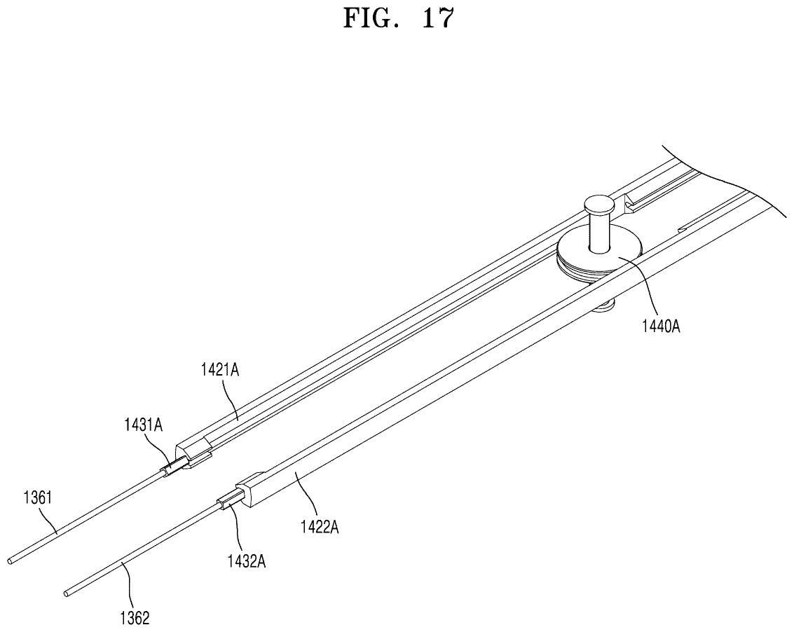

FIGS. 16 and 17 are perspective views illustrating some components of the yaw power transmission part 1300A according to an embodiment of the present disclosure.

Referring to FIGS. 16 and 17, the yaw power transmission part 1300A may include the yaw drive wires 1361 and 1362, a first yaw power transmission unit 1310A, and a second yaw power transmission unit 1320A. Further, the connection part 1400 may include a first wire coupling member 1421A, a second wire coupling member 1422A, a first wire fixing member 1431A, a second wire fixing member 1432A, and a direction-changing member 1440A.

The yaw drive wires 1361 and 1362 are moved by the yaw drive motor 1511 and may control the yaw motion of the end tool 1100. The yaw drive wires may have a first yaw drive wire 1361 and a second yaw drive wire 1362, and the end tool 1100 may be yaw-rotated in one direction by the first yaw drive wire 1361 or yaw-rotated in another direction by the second yaw drive wire 1362. That is, the first yaw drive wire 1361 and the second yaw drive wire 1362 may control the yaw rotation of the end tool 1100 while moving linearly, and at this time, the first yaw drive wire 1361 and the second yaw drive wire 1362 may move in opposite directions while maintaining tension.\

The first yaw power transmission unit 1310A may control the movement of the first yaw drive wire 1361.

The first yaw power transmission unit 1310A may include a first lead screw 1311A and a first linear movement guide 1312A.

The first lead screw 1311A may be rotated by the driving of the yaw drive motor 1511.

Specifically, a main gear 15112 may be disposed on the yaw drive motor 1511. The power generation part 1500 may include the main gear 15112 on one side of the yaw drive motor 1511, so that the main gear 15112 can rotate in response to the driving of the yaw drive motor 1511.

The first yaw power transmission unit 1310A may further include a first sub-gear 13111A that is engaged with the main gear 15112. One end of the first lead screw 1311A may be fixed to the first sub-gear 13111A. When the yaw drive motor 1511 is driven, the main gear 15112 may rotate, and as the first sub-gear 13111A engaged with the main gear 15112 rotates, the first lead screw 1311A may rotate.

The first lead screw 1311A may include a first thread 13112A. As the first thread 13112A is formed on an outer surface of the first lead screw 1311A, the first lead screw 1311A may be threadedly engaged with the first linear movement guide 1312A.

The first linear movement guide 1312A may be threadedly engaged with the first lead screw 1311A. As the first lead screw 1311A rotates, the first linear movement guide 1312A may move linearly in the longitudinal direction.

The first linear movement guide 1312A may include a first screw nut 13121A and a first contact guide 13122A.

The first screw nut 13121A may linearly move while being threadedly engaged with the first lead screw 1311A. The first screw nut 13121A may be formed in various shapes and types of members that may be fitted onto the first lead screw 1311A and may linearly move in response to the rotation of the first lead screw 1311A.

The first contact guide 13122A may be fixed to the first screw nut 13121A, and may move linearly together with the first screw nut 13121A. The first contact guide 13122A may have one end in contact with the first wire coupling member 1421A and another end fixed to the first screw nut 13121A. Accordingly, when the first lead screw 1311A rotates, the first contact guide 13122A may linearly move together with the first screw nut 13121A and push the first wire coupling member 1421A in one direction (the arrow A1 direction). The first wire coupling member 1421A is positioned inside the shaft 1410, and may be coupled to the first yaw drive wire 1361.

The first wire coupling member 1421A is coupled to the first yaw drive wire 1361, and may come into contact with the first linear movement guide 1312A. The first wire coupling member 1421A may have one end to which the first yaw drive wire 1361 is fixed and another end in contact with the first contact guide 13122A. Accordingly, when the first contact guide 13122A pushes the first wire coupling member 1421A in one direction (the arrow A1 direction), the first yaw drive wire 1361 may move in one direction (the arrow A1 direction) together with the first wire coupling member 1421A.

The first wire fixing member 1431A may fasten the first yaw drive wire 1361 to the first wire coupling member 1421A. The first wire fixing member 1431A may be formed of various members capable of fastening the first yaw drive wire 1361 to the first wire coupling member 1421A, so that the first yaw drive wire 1361 is fixed to the first wire coupling member 1421A and can move linearly together with the first wire coupling member 1421A.

As such, the first yaw power transmission unit 1310A may be connected to the first yaw drive wire 1361, and may linearly move the first yaw drive wire 1361. The main gear 15112 may be positioned on one side of the yaw drive motor 1511, and when the first sub-gear 13111A is engaged with the main gear 15112 and rotates, the first lead screw 1311A may rotate. When the first lead screw 1311A rotates, the first screw nut 13121A, which is threadedly engaged with the first lead screw 1311A, linearly moves, which causes the first contact guide 13122A to push the first wire coupling member 1421A and move linearly. As a result, the first yaw drive wire 1361 connected to the first wire coupling member 1421A may move linearly.

The second yaw power transmission unit 1320A may control the movement of the second yaw drive wire 1362.

The second yaw power transmission unit 1320A may include a second lead screw 1321A and a second linear movement guide 1322A.

The second lead screw 1321A may be rotated by the driving of the yaw drive motor 1511.

Specifically, the main gear 15112 may be positioned on the yaw drive motor 1511. The power generation part 1500 may include the main gear 15112 on one side of the yaw drive motor 1511, so that the main gear 15112 can rotate in response to the driving of the yaw drive motor 1511.

The second yaw power transmission unit 1320A may further include a second sub-gear 13211A that is engaged with the main gear 15112. One end of the second lead screw 1321A may be fixed to the second sub-gear 13211A. When the yaw drive motor 1511 is driven, the main gear 15112 may rotate, and as the second sub-gear 13211A engaged with the main gear 15112 rotates, the second lead screw 1321A may rotate.

In an embodiment, the second lead screw 1321A may be positioned on the opposite side of the first lead screw 1311A with respect to the yaw drive motor 1511. In other words, the second sub-gear 13211A may be positioned on the opposite side of the first sub-gear 13111A with respect to the main gear 15112. Referring to FIG. 10 again, the first sub-gear 13111A may be positioned on one side of the main gear 15112, and the second sub-gear 13211A may be positioned on another side of the main gear 15112.

In a structure in which gears are engaged, backlash occurs due to meshing between gear teeth. When the backlash is excessively large, excessive rotational resistance may occur when a torque direction of the yaw drive motor 1511 changes, and gear teeth may wear, thereby leading to vibration or noise issues. In the yaw power transmission part 1300A of the present disclosure, when the main gear 15112, the first sub-gear 13111A, and the second sub-gear 13211A are positioned in sequence, the effect of backlash may accumulate in the second sub-gear 13211A due to the rotation of the main gear 15112. In this case, when the yaw drive motor 1511 rotates, the first lead screw 1311A and the second lead screw 1321A do not rotate in a one-to-one correspondence but instead rotate with different angular deformations. As a result, an issue may arise in which excessively large or small tension is applied to one of the first yaw drive wire 1361 and the second yaw drive wire 1362.

For example, in the power transmission part 1300 according to an embodiment of the present disclosure, the first lead screw 1311A and the second lead screw 1321A may be positioned on opposite sides of the yaw drive motor 1511, and the first sub-gear 13111A, the main gear 15112, and the second sub-gear 13211A may be positioned in this order. Through this arrangement, the power transmission part 1300 of the present disclosure may offset the backlash of the first sub-gear 13111A and the second sub-gear 13211A against each other, thereby improving structural stability and usability.

The second lead screw 1321A may include a second thread 13212A. As the second thread 13212A is formed on an outer surface of the second lead screw 1321A, the second lead screw 1321A may be threadedly engaged with the second linear movement guide 1322A.

In an embodiment, the first thread 13112A of the first lead screw 1311A and the second thread 13212A of the second lead screw 1321A may be formed in opposite directions.

Specifically, when the first lead screw 1311A and the second lead screw 1321A are disposed with the yaw drive motor 1511 disposed in between, the first lead screw 1311A and the second lead screw 1321A may rotate in the same direction in response to the rotation of the yaw drive motor 1511. The first yaw drive wire 1361 and the second yaw drive wire 1362 move in opposite directions to control the yaw rotation of the end tool 1100. Accordingly, the first yaw power transmission unit 1310A and the second yaw power transmission unit 1320A must be driven in opposite directions. Accordingly, in the yaw power transmission part 1300A of the present disclosure, since the first thread 13112A and the second thread 13212A are formed in opposite directions, the first linear movement guide 1312A and the second linear movement guide 1322A may move in opposite directions.

That is, the yaw power transmission part 1300A of the present disclosure may offset the influence of backlash according to the gear structure by positioning the first lead screw 1311A and the second lead screw 1321A on opposite sides of the yaw drive motor 1511. At the same time, in the yaw power transmission part 1300A of the present disclosure, the first thread 13112A of the first lead screw 1311A and the second thread 13212A of the second lead screw 1321A are formed in opposite directions, thereby allowing the first yaw power transmission unit 1310A and the second yaw power transmission unit 1320A to be driven in opposite directions in response to the rotation of the yaw drive motor 1511.

The second linear movement guide 1322A may be threadedly engaged with the second lead screw 1321A. As the second lead screw 1321A rotates, the first linear movement guide 1312A may move linearly in the longitudinal direction.

The second linear movement guide 1322A may include a second screw nut 13221A and a second contact guide 13222A.

The second screw nut 13221A may linearly move while being threadedly engaged with the second lead screw 1321A. The second screw nut 13221A may be formed in various shapes and types of members that may be fitted onto the second lead screw 1321A and may linearly move in response to the rotation of the second lead screw 1321A.

The second contact guide 13222A is fixed to the second screw nut 13221A and may move linearly together with the second screw nut 13221A. The second contact guide 13222A may have one end in contact with the second wire coupling member 1422A, and another end fixed to the second screw nut 13221A. Accordingly, when the second lead screw 1321A rotates, the second contact guide 13222A moves linearly may linearly move together with the second screw nut 13221A and push the second wire coupling member 1422A in one direction (the arrow A1 direction).

The second wire coupling member 1422A may be positioned inside the shaft 1410 and coupled to the second yaw drive wire 1362.

The second wire coupling member 1422A may be coupled to the second yaw drive wire 1362 and may come into contact with the second linear movement guide 1322A. The second wire coupling member 1422A have one end to which the second yaw drive wire 1362 is fixed and another end in contact with the second contact guide 13222A. Accordingly, when the second contact guide 13222A pushes the second wire coupling member 1422A in one direction (the arrow A1 direction), the second yaw drive wire 1362 may move in one direction (the arrow A1 direction) together with the second wire coupling member 1422A.

The second wire fixing member 1432A may fasten the second yaw drive wire 1362 to the second wire coupling member 1422A. The second wire fixing member 1432A may be formed of various members capable of fastening the second yaw drive wire 1362 to the second wire coupling member 1422A, so that the second yaw drive wire 1362 is fixed to the second wire coupling member 1422A and can move linearly together with the second wire coupling member 1422A.

In this way, the second yaw power transmission unit 1320A may be connected to the second yaw drive wire, and may linearly move the second yaw drive wire 1362. The main gear 15112 may be positioned on one side of the yaw drive motor 1511, and when the second sub-gear 13211A is engaged with the main gear 15112 and rotates, the second lead screw 1321A may rotate. As the second lead screw 1321A rotates, the second screw nut 13221A, which is threadedly engaged with the second lead screw 1321A, moves linearly, which causes the second contact guide 13222A to push the second wire coupling member 1422A and move linearly. As a result, the second yaw drive wire 1362 connected to the second wire coupling member 1422A may move linearly.

Further, the second yaw power transmission unit 1320A may improve issues, such as rotational resistance due to backlash in the gear structure and gear tooth wear by positioning the second lead screw 1321A in the opposite direction to the first lead screw 1311A with respect to the yaw drive motor 1511. At this time, as the first thread 13112A of the first lead screw 1311A and the second thread 13212A of the second lead screw 1321A are formed in opposite directions, the first linear movement guide 1312A and the second linear movement guide 1322A may move in opposite directions according to the driving of the yaw drive motor 1511.

The direction-changing member 1440A may maintain the tension of the drive wire 1360 while controlling the movement directions.

The direction-changing member 1440A may be positioned between the first wire coupling member 1421A and the second wire coupling member 1422A in the shaft 1410, and may control movement directions of the first yaw drive wire 1361 and the second yaw drive wire 1362.

When the yaw drive motor 1511 rotates, the first contact guide 13122A pushes the first wire coupling member 1421A, thereby allowing the first yaw drive wire 1361 to move linearly in one direction (the arrow A1 direction). At this time, since the second contact guide 13222A is in face-to-face contact with the second wire coupling member 1422A, when the second wire coupling member 1422A moves in another direction (the arrow A2 direction) together with the second contact guide 13222A, a structure is required to maintain the tension of the first yaw drive wire 1361 and the second yaw drive wire 1362.

Likewise, when the yaw drive motor 1511 rotates in the opposite direction, the second contact guide 13222A may push the second wire coupling member 1422A, thereby allowing the second yaw drive wire 1362 to move linearly in one direction (the arrow A1 direction). In some embodiments, since the first contact guide 13122A is in face-to-face contact with the first wire coupling member 1421A, when the first wire coupling member 1421A moves in another direction (the arrow A2 direction) together with the first contact guide 13122A, a structure is required to maintain the tension of the first yaw drive wire 1361 and the second yaw drive wire 1362.

The surgical instrument 1000 of the present disclosure controls the first yaw drive wire 1361 and the second yaw drive wire 1362 to move in opposite directions by the same displacement through the direction-changing member 1440A, thereby allowing the first yaw drive wire 1361 and the second yaw drive wire 1362 to maintain a constant tension.

In an embodiment, as shown in FIG. 17, the connection part 1400 may have an auxiliary pulley as the direction-changing member 1440A. At this time, the first yaw drive wire 1361 and the second yaw drive wire 1362 may extend to the end tool 1100 while being wound around the auxiliary pulley 1440A. That is, as shown in FIG. 11, the first yaw drive wire 1361 and the second yaw drive wire 1362 may form a single loop while being wound around a yaw driving pulley 1301A and the direction-changing member 1440A.

As the connection part 1400 includes the direction-changing member 1440A as an auxiliary pulley, the auxiliary pulley 1440A may rotate according to the linear movement of one of the first yaw drive wire 1361 and the second yaw drive wire 1362, thereby causing another one of the first yaw drive wire 1361 and the second yaw drive wire 1362 to move linearly. At this time, since the tension of the first yaw drive wire 1361 and the second yaw drive wire 1362 can be kept constant while being wound around the auxiliary pulley 1440A, the first yaw drive wire 1361 and the second yaw drive wire 1362 may move in opposite directions by the same displacement.

That is, as shown in FIG. 15, direction-changing members 1440A, 1440B, and 1440C may be disposed with the yaw drive wires 1361 and 1362, the pitch drive wires 1363 and 1364, and the firing drive wires 1365 and 1366 wound therearound, respectively. Each pair of drive wires 1360 may be coupled to the respective wire coupling members 1421 and 1422 and may move linearly. In particular, each pair of drive wires 1360 may move in opposite directions while maintaining tension by the direction-changing member 1440, thereby controlling the operation of the end tool 1100.

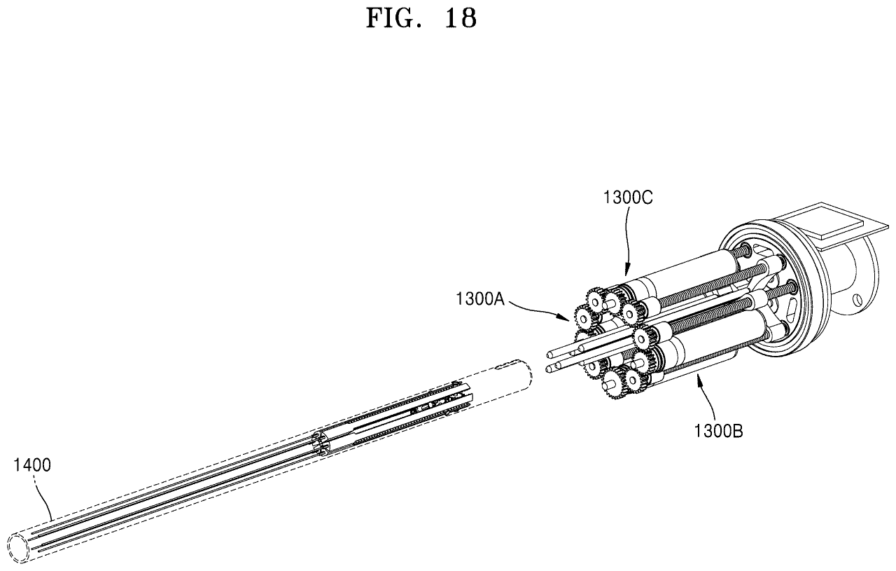

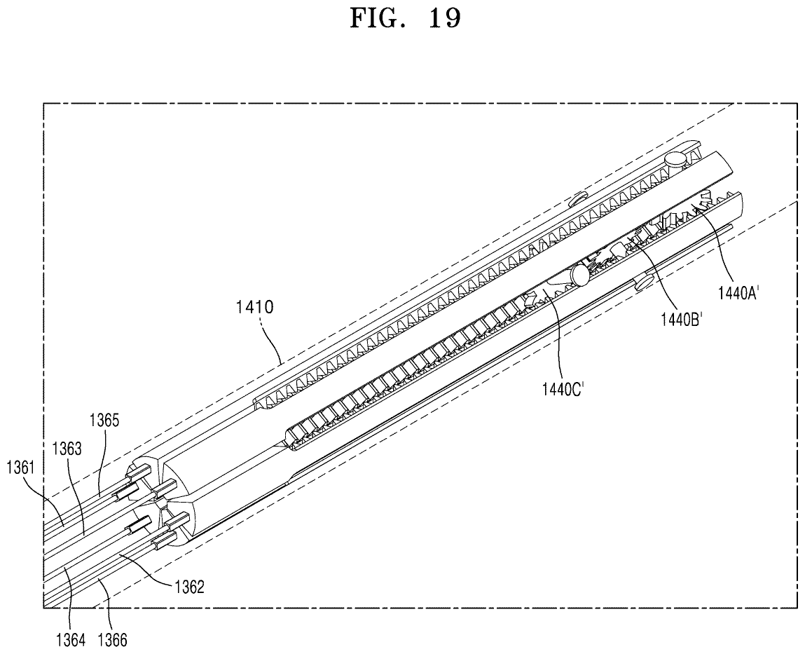

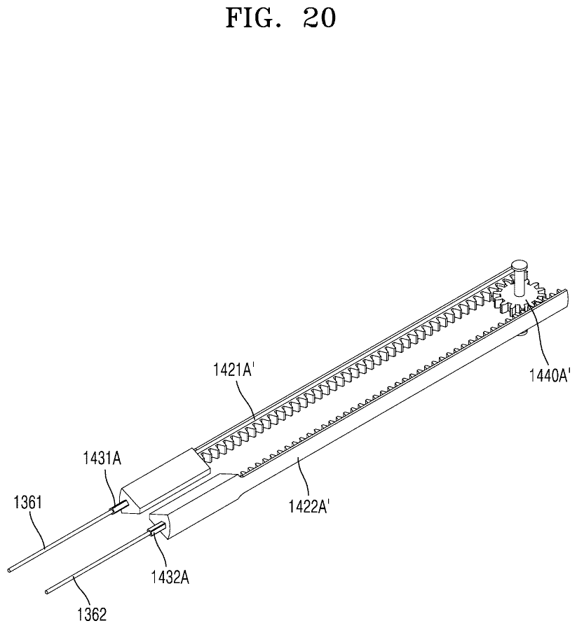

FIGS. 18 and 19 are perspective views illustrating a plurality of power transmission parts 1300 provided in a surgical instrument 1000 according to another embodiment of the present disclosure. FIG. 20 is a perspective view illustrating some components of a yaw power transmission part 1300A of FIG. 18.

The surgical instrument 1000 according to the embodiment of FIGS. 18 to 20 is different from the embodiment of FIGS. 14 to 17 in terms of a detailed structure of a direction-changing member 1440 included in a connection part 1400 and corresponding wire coupling members 1421 and 1422, and thus, hereinafter, a description will be provided focusing on these differences.

Referring to FIGS. 18 to 20, the connection part 1400 of the surgical instrument 1000 according to another embodiment of the present disclosure may have the auxiliary gear as a direction-changing member 1440A′. At this time, a first wire coupling member 1421A′ and a second wire coupling member 1422A′ may each have gear teeth that engage with the auxiliary gear 1440A′.

Specifically, first gear teeth may be formed on an inner side of the first wire coupling member 1421A′, and may have one end to which the first yaw drive wire 1361 is fixed. Further, second gear teeth may be formed on an inner side of the second wire coupling member 1422A′, and may have one end to which the second yaw drive wire 1362 is fixed.

The auxiliary gear 1440A′ may be positioned so as to be engaged with the first gear teeth of the first wire coupling member 1421A′ and the second gear teeth of the second wire coupling member 1422A′. Accordingly, as one of the first wire coupling member 1421A′ and the second wire coupling member 1422A′ moves linearly in one direction, the auxiliary gear 1440A′ may rotate, thereby allowing another one of the first wire coupling member 1421A′ and the second wire coupling member 1422A′ to move linearly in the opposite direction.

That is, as shown in FIG. 19, the surgical instrument 1000 may include auxiliary gears as direction-changing members 1440A′, 1440B′, and 1440C′, respectively. When gear teeth are formed on each of the wire coupling members 1421A′ and 1422A′, the drive wire 1360 may be fixedly coupled to one end of each of the wire coupling member 1421A′ and 1422A′. At this time, the method by which the drive wire 1360 is fixed to each of the wire coupling members 1421A′ and 1422A′ is not particularly limited. Each pair of drive wires 1360 may be coupled to the wire coupling members 1421S′ and 1422S′ and may move linearly. In particular, each pair of drive wires 1360 may move in opposite directions while maintaining tension by the direction-changing member 1440A′, thereby controlling the operation of the end tool 1100.

The above has described the method of supplying power to the yaw drive wires 1361 and 1362 to yaw-rotate the end tool 1100, with a focus on the yaw power transmission part 1300A of the surgical instrument 1000. As described above, the configuration and driving method of the yaw power transmission part 1300A may be substantially the same as those applied to the pitch power transmission part 1300B and the firing power transmission part 1300C. Accordingly, the configuration and driving method of the pitch power transmission part 1300B and the firing power transmission part 1300C will be described by referencing the descriptions provided for the yaw power transmission part 1300A.

Further, in the present specification, the embodiment has been described in which the surgical instrument 1000 includes the yaw drive motor 1511, the pitch drive motor 1512, and the firing drive motor 1513, and the power transmission parts 1300A, 1300B, and 1300C are provided for the respective drive motors 1511, 1512, and 1513. However, the present disclosure is not limited thereto, and the number, arrangement, and structure of the power transmission part 1300 included in the surgical instrument 1000 may be variously modified.

For example, when the end tool 1100 of the surgical instrument 1000 performs only yaw and pitch motions without performing a firing motion, the surgical instrument 1000 may include only the yaw power transmission part 1300A and the pitch power transmission part 1300B.

In the surgical instrument according to an embodiment of the present disclosure, power generated by drive motors may be transmitted to an end tool through power transmission parts. Each of the power transmission parts may include a power transmission unit capable of linear movement by the driving of the motor, such that a linear movement guide of the power transmission unit may move linearly and push wires. At this time, movement directions of the wires may be switched by a direction-changing member, and as the wires are pulled, the operation of the end tool may be controlled. Further, in the surgical instrument according to an embodiment of the present disclosure, the end tool and a motor pack may rotate together, thereby preventing the wires from twisting inside and enabling unlimited roll rotation.

The power transmission part of the surgical instrument according to an embodiment of the present disclosure may include a lead screw that is rotatable by the driving of the motor and the linear movement guide that is capable of linear movement in response to the rotation of the lead screw. In particular, the power transmission unit may be provided for each drive wire, and a pair of power transmission units for a pair of drive wires that control a single operation may be positioned on opposite sides of the drive motor. This configuration may prevent issues such as tension loss and wear caused by backlash in a gear during power transmission. At the same time, by varying directions of threads formed on the lead screws, the pair of power transmission units may move in opposite directions, and the drive wire may move while maintaining tension due to the direction-changing member.

In a surgical instrument according to an embodiment of the present disclosure, power generated by a drive motor can be transmitted to an end tool through a power transmission part. The power transmission part includes a linear movement guide, which is threadedly engaged with a lead screw, as a power transmission unit, thereby enabling a wire to move linearly in response to a rotation of a motor. At this time, the wire can be pulled while maintaining tension by a direction-changing member, thereby controlling operations of the end tool. Further, a pair of power transmission units are positioned on opposite sides of the drive motor, and by varying only the shape of threads on the lead screw, the influence of backlash caused by a gear structure can be minimized while allowing the pair of power transmission units to move in opposite directions.

In a surgical instrument according to an embodiment of the present disclosure, an end tool and a motor pack can roll-rotate together, thereby preventing an issue of wire twisting inside and enabling unlimited roll rotation.

The present disclosure has been described above with a focus on embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

What is claimed is:

1. A surgical instrument comprising:
an end tool configured to be rotatable in at least one direction;
a drive wire having a first wire and a second wire that control the rotation of the end tool;
a power generation part having at least one motor configured to generate a power to drive the end tool; and
a power transmission part connected to the end tool and configured to transmit the power generated by the power generation part to the end tool,
wherein the power transmission part includes:
a first power transmission unit configured to linearly move the first wire in a longitudinal direction of the first wire by driving of the at least one motor; and
a second power transmission unit configured to linearly move the second wire in a longitudinal direction of the second wire by the driving of the at least one motor.

2. The surgical instrument of claim 1, wherein, when the at least one motor is driven, the first wire and the second wire are linearly moved in opposite directions by the first power transmission unit and the second power transmission unit.

3. The surgical instrument of claim 2, wherein
the first power transmission unit includes:
a first lead screw configured to be rotated by the driving of the at least one motor and having a first thread; and
a first linear movement guide connected to the first wire, threadedly engaged with the first lead screw, and configured to move linearly in response to the rotation of the first lead screw, and
the second power transmission unit includes:
a second lead screw configured to be rotated by the driving of the at least one motor and having a second thread; and
a second linear movement guide connected to the second wire, threadedly engaged with the second lead screw, and configured to move linearly in response to the rotation of the second lead screw.

4. The surgical instrument of claim 3, wherein
the power generation part includes a main gear coupled to the at least one motor and configured to be rotatable,
the first power transmission unit further includes a first sub-gear, to which one end of the first lead screw is connected and which is engaged with the main gear at one side of the main gear, and
the second power transmission unit further includes a second sub-gear, to which one end of the second lead screws is connected and which is engaged with the main gear at another side of the main gear.

5. The surgical instrument of claim 4, wherein the first thread and the second thread are formed in opposite directions.

6. The surgical instrument of claim 3, further comprising a connection part positioned between the power generation part and the end tool and having a shaft in which the drive wire is accommodated,
wherein the connection part includes:
a first wire coupling member having one end connected to the first wire and configured to move linearly while maintaining contact between the other end and the first linear movement guide; and
a second wire coupling member having one end connected to the second wire and configured to move linearly while maintaining contact between the other end and the second linear movement guide

7. The surgical instrument of claim 6, wherein the connection part further includes a direction-changing member positioned between the first wire coupling member and the second wire coupling member and configured to switch movement directions of the first wire and the second wire.

8. The surgical instrument of claim 7, wherein
the direction-changing member is provided as an auxiliary pulley, and
the first wire and the second wire extend to the end tool while being wound around the auxiliary pulley.

9. The surgical instrument of claim 7, wherein
the first wire coupling member has one end fixed to the first wire and includes first gear teeth formed in a longitudinal direction of the first wire coupling member,
the second wire coupling member has one end fixed to the second wire and includes second gear teeth formed in a longitudinal direction of the second wire coupling member, and
the direction-changing member is provided as an auxiliary gear engaged with the first gear teeth and the second gear teeth.

10. The surgical instrument of claim 6, wherein the connection part further includes:
a first wire fixing member fixed to the first wire and configured to connect the first wire coupling member to the first wire; and
a second wire fixing member fixed to the second wire and configured to connect the second wire coupling member to the second wire.

11. The surgical instrument of claim 2, wherein the power generation part includes a motor pack having a motor housing and a plurality of drive motors positioned inside the motor housing,
wherein the motor pack includes:
a pitch drive motor configured to generate a power to enable the end tool to perform a pitch motion; and
a yaw drive motor configured to generate a power to enable the end tool to perform a yaw motion.

12. The surgical instrument of claim 11, wherein the power generation part further includes a roll rotation unit configured to roll-rotate the motor pack,
wherein the roll rotation unit includes:
a roll drive motor configured to generate a power to roll-rotate the motor pack;
a roll drive gear connected to the roll drive motor; and
a rotation guide unit connected to the motor housing, engaged with the roll drive gear, and configured to roll-rotate the motor pack by driving of the roll drive motor.

13. The surgical instrument of claim 11, wherein the power transmission part further includes
a yaw power transmission part configured to control a movement of a yaw drive wire by driving of the yaw drive motor; and
a pitch power transmission part configured to control a movement of a pitch drive wire by driving of the pitch drive motor.

14. The surgical instrument of claim 13, wherein
the end tool includes an operation member moving in a longitudinal direction of the end tool, and
the motor pack further includes a firing drive motor configured to generate a power to linearly move the operation member.

15. The surgical instrument of claim 14, wherein the power transmission part further includes a firing power transmission part configured to control a movement of a firing drive wire by driving of the firing drive motor.

16. A handle of a surgical instrument comprising:
a power generation part having at least one motor that generates a power to drive an end tool configured to be rotatable in at least one direction; and
a power transmission part configured to linearly move a first wire and a second wire, which control the rotation of the end tool, and transmit the power generated by the power generation part to the end tool,
wherein the power transmission part includes:
a first power transmission unit configured to linearly move the first wire in a longitudinal direction of the first wire by driving of the at least one motor; and
a second power transmission unit configured to linearly move the second wire in a longitudinal direction of the second wire by the driving of the at least one motor.

17. The handle of claim 16, wherein when the at least one motor is driven, the first wire and the second wire are linearly moved in opposite directions by the first power transmission unit and the second power transmission unit.

18. The handle of claim 17, wherein
the first power transmission unit includes:
a first lead screw configured to be rotated by the driving of the at least one motor and having a first thread; and
a first linear movement guide connected to the first wire, threadedly engaged with the first lead screw, and configured to move linearly in response to the rotation of the first lead screw, and
the second power transmission unit includes:
a second lead screw configured to be rotated by the driving of the at least one motor and having a second thread; and
a second linear movement guide connected to the second wire, threadedly engaged with the second lead screw, and configured to move linearly in response to the rotation of the second lead screw.

19. A connection part of a surgical instrument, the connection part comprising:
a shaft positioned between an end tool configured to be rotatable in at least one direction and a power generation part configured to provide a power to the end tool, the shaft accommodating a first wire and a second wire that control the rotation of the end tool;
a first wire coupling member positioned inside the shaft and having one end connected to the first wire; and
a second wire coupling member positioned inside the shaft and having one end connected to the second wire,
wherein the first wire coupling member and the second wire coupling member
linearly move the first wire and the second wire in opposite directions while being linearly moved by driving of a motor of the power generation part.

20. The connection part of claim 19, further comprising a direction-changing member positioned between the first wire coupling member and the second wire coupling member and configured to switch movement directions of the first wire and the second wire.
